(19) United States
(12) Patent Application Publication
(10) Pub. No.: US 2026/0345686 A1
(43) Pub. Date: Nov. 5, 2026

(54) COMPOSITIONS AND SYSTEMS FOR COMBINATORIAL THERAPIES CONTAINING FUCOSYLATED STEM CELLS AND HEMATOPOIETIC ENHANCERS AND/OR IMMUNOTHERAPEUTICS AND METHODS OF PRODUCTION AND USE THEREOF

(71) Applicant: Targazyme, Inc., Carlsbad, CA (US)

(72) Inventors: David John KYLE, Davis, CA (US); Reid P. BISSONNETTE, Carlsbad, CA (US); Lynnet KOH, Carlsbad, CA (US); Gopalakrishnan RAMAKRISHNAN, Chicago, IL (US)

(21) Appl. No.: 19/163,112

(22) PCT Filed: Mar. 8, 2024
(86) PCT No.: PCT/US24/19151

§ 371 Date: Sep. 8, 2025

Related U.S. Application Data
(60) Provisional application No. 63/489,108, filed on Mar. 8, 2023.

(51) Int. Cl. A61K 35/28; A61K 38/18; A61K 38/19; A61K 39/395; A61P 7/00; A61P 35/00

(52) U.S. Cl. CPC A61K 35/28; A61K 38/1816; A61K 38/193; A61K 38/196; A61K 39/3955; A61P 7/00; A61P 35/00

(57) ABSTRACT

Compositions, medicaments, kits, and systems are disclosed that include surface-modified stem cells and hematopoietic enhancers and/or monoclonal antibodies for hematopoietic stem cell transplant therapy. The stem cells are ex vivo fucosylated and may be autologous or allogeneic. The hematopoietic enhancers may be white cell enhancers, red cell enhancers, and/or platelet enhancers. Methods of producing and/or using the compositions, medicaments, kits, and systems are also disclosed. Methods of increasing engraftment of hematopoietic stem cells and myeloid lineage cells upon hematopoietic stem cell therapy are also disclosed.

### CROSS REFERENCE TO RELATED APPLICATIONS/INCORPORATION BY REFERENCE STATEMENT

The subject application claims benefit under 35 USC § 119(e) of U.S. Provisional Application No. 63/489,108, filed Mar. 8, 2023. The entire contents of the above-referenced patent application(s) are hereby expressly incorporated herein by reference.

### REFERENCE TO SEQUENCE LISTING SUBMITTED ELECTRONICALLY

The instant application contains, as a separate part of the present disclosure, a Sequence Listing which has been submitted via EFS-Web in computer readable form as an XML file. The Sequence Listing, created Mar. 4, 2024, is named “1001080wo Sequence Listing.xml” and is 2,289 bytes in size. The entire contents of the Sequence Listing are hereby incorporated herein by reference.

### BACKGROUND

Stem cells are undifferentiated cells from which all other cells with specialized functions in our body are generated. Under the right conditions in the body or a laboratory, stem cells divide to form more cells called daughter cells. These daughter cells become either new stem cells or differentiate into specialized cells with a more specific function, such as blood cells, brain cells, heart muscle cells, bone cells and cells in our immune system. No other cell in the body has the natural ability to generate new cell types. Hematopoietic stem cells have been used to treat various types of blood-borne cancers, including leukemia, lymphoma, and multiple myeloma, as well as other diseases such as blood disorders, genetic diseases, and metabolic disorders. In these cases, chemotherapy or radiation therapy are used to kill cancer cells or clear the body of diseased cells or healthy cells to enable stem cells to better engraft and regenerate; however, this treatment also destroys the healthy stem cells and various other cells. The Hematopoietic Stem Cell Transplant (HSCT) replaces the destroyed stem cells with new stem cells and helps the body recover. There are two main types of stem cell transplants: autologous and allogeneic. Autologous transplants involve using the patient's own stem cells, while allogeneic transplants involve using stem cells from a donor.

For a significant period of time during the HSCT process the patient is severely immunocompromised and many patients succumb to the lethal consequences of this process even though they may have no more underlying disease. Key problems encountered in the immunocompromised phase of the HSCT process include: elevated risks of bacterial, viral and fungal infection; internal uncontrolled bleeding; graft failure (GF); graft vs host disease (GvHD); and even growth of cancers due to lack of immune cells to combat the cancers.

Shortening the duration of the time when the patients are immuno-compromised can significantly improve patient outcomes but it takes time to move the newly introduced stem cells out of the circulatory system into the tissues, and ultimately into the bone marrow where they will engraft and start the process of rebuilding the immune system. One solution that has been proposed is the ex vivo fucosylation of the stem cell surfaces (Popat, 2015, and US Patent Application Publication Nos. 2011/0091434, 2014/0161782, 2017/0058261, and 2019/0017023 all incorporated herein by reference in their entirety). This fucosylation has been shown to improve the trafficking of the stem cells out of the circulatory system and into the bone marrow, where they will differentiate into new immune cells. However, this process involves a one-way modification of the stem cells before they are infused into the patient, and as these stem cells divide, the fucosylation on the cell surface becomes distributed to daughter cells and thereby diluted.

### BRIEF DESCRIPTION OF THE DRAWINGS

FIG. 1 contains a 3-D rendition of FUT6 showing the hydrophobic tail that anchors the protein in a cell membrane. Amino acid sequence has been assigned SEQ ID NO:1.

FIG. 2 graphically illustrates that fucosylation enhances both the rate and amount of engraftment of hematopoietic stem cells after transplantation.

FIG. 3 graphically illustrates that fucosylation enhances engraftment of all mononuclear cells. Changes are shown as percentages of engraftment.

FIG. 4 graphically illustrates that the enhanced engraftment of FIG. 3 was observed in all cell lineages.

FIG. 5 graphically illustrates that fucosylation induces a change in the composition of engrafted cells and cell composition in mammals.

FIG. 6 graphically illustrates that fucosylation enhances relative engraftment of the myeloid and lymphoid lineage.

FIG. 7 graphically illustrates that the concomitant treatment of fucosylated stem cells transplanted along with a hematopoietic enhancer (PEGfilgrastim) reduced the number of episodes of infection in patients after completion of HSCT, compared to historical controls.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, and therapeutic applications.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, systems, kits, and methods disclosed herein can be made and executed without undue experimentation in light of the present disclosure. While the compositions, systems, kit, and methods have been described in terms of particular embodiments, it will be apparent to those of skill in the art that variations may be applied to the compositions, systems, kits, and methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

The use of ordinal number terminology (i.e., “first,” “second,” “third,” “fourth,” etc.) is solely for the purpose of differentiating between two or more items and, unless explicitly stated otherwise, is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example (but not by way of limitation), when associated with a particular event or circumstance, the term “substantially” means that the subsequently described event or circumstance occurs at least 80% of the time, or at least 85% of the time, or at least 90% of the time, or at least 95% of the time. In addition, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with,” “coupled to,” and “connected to” include both direct association/coupling/binding of two moieties to one another as well as indirect association/coupling/binding of two moieties to one another. When two moieties are indirectly associated/coupled/connected to one another, one or more intervening elements may be present therebetween (e.g., a spacer, linking moiety, etc.). Non-limiting examples of associations/couplings/bindings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “pharmaceutically acceptable” refers to compounds and compositions which are suitable for administration to humans and/or animals without undue adverse side effects, such as (but not limited to) toxicity, irritation, and/or allergic response, commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue. Non-limiting examples include a human, bovine, rat, mouse, dog, monkey, ape, goat, sheep, cow, or deer.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent to a subject/patient for therapeutic and/or prophylactic/preventative purposes.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

A “therapeutic composition” or “pharmaceutical composition” refers to an agent that may be administered in vivo to bring about a therapeutic and/or prophylactic/preventative effect.

The term “regimen” refers to a protocol for dosing and timing the administration of one or more therapies (e.g., combinations described herein or another active agent such as an anti-cancer agent described herein) for treating a disease, disorder, or condition described herein. A regimen can include periods of active administration and periods of rest as known in the art. Active administration periods include administration of combinations and compositions described herein and the duration of time of efficacy of such combinations and compositions. Rest periods of regimens described herein include a period of time in which no compound is actively administered, and in certain instances, includes time periods where the efficacy of such compounds can be minimal. Combination of active administration and rest in regimens described herein can increase the efficacy and/or duration of administration of the combinations and compositions described herein.

The terms “therapies” and “therapy” refer to any protocol(s), method(s), and/or agent(s) that can be used in the prevention, treatment, management, and/or amelioration of a disease, disorder, or condition or one or more symptoms thereof. In certain instances, the term refers to active agents such as an anti-cancer agent described herein. The term “therapy” can refer to anti-viral therapy, anti-bacterial therapy, anti-fungal therapy, anti-cancer therapy, biological therapy, supportive therapy, and/or other therapies useful in treatment, management, prevention, or amelioration of a disease, disorder, or condition or one or more symptoms thereof known to one skilled in the art, for example, a medical professional such as a physician.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or sufficient to exhibit a detectable therapeutic effect without undue adverse side effects (such as (but not limited to) toxicity, irritation, and allergic response) commensurate with a reasonable benefit/risk ratio when used in the manner of the present disclosure. The therapeutic effect may include, for example but not by way of limitation, preventing, inhibiting, or reducing the occurrence of infection by or growth of microbes and/or opportunistic infections. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the disease/infection in conjunction with the compositions of the present disclosure. This concurrent therapy can be sequential therapy, where the patient is treated first with one composition and then the other composition, or the two compositions are given simultaneously.

The term “administering” refers to the act of delivering a combination or composition described herein into a subject by such routes as oral, mucosal, topical, suppository, intravenous, parenteral, intraperitoneal, intramuscular, intralesional, intrathecal, intranasal or subcutaneous administration. Parenteral administration includes intravenous, intramuscular, intra-arteriole, intradermal, subcutaneous, intraperitoneal, intraventricular, and intracranial administration. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition).

The terms “administration” and “administering,” as used herein, will be understood to include all routes of administration known in the art. In addition, the compositions of the present disclosure (and/or the methods of administration of same) may be designed to provide delayed, controlled, or sustained release using formulation techniques which are well known in the art.

The term “coadministration” refers to administration of two or more agents (e.g., a combination described herein and another active agent such as an anti-cancer agent described herein). The timing of coadministration depends in part of the combination and compositions administered and can include administration at the same time, just prior to, or just after the administration of one or more additional therapies, for example cancer therapies such as chemotherapy, hormonal therapy, radiotherapy, or immunotherapy. The composition(s) of the present disclosure can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compounds described herein can be used in combination with one another, with other active agents known to be useful in treating cancer.

The term “pharmaceutically acceptable carrier or excipient” includes any carriers or excipients known in the art may be utilized in accordance with the present disclosure. For example (but not by way of limitation), a physiological compatible carrier (e.g., saline) that is compatible with maintaining the structure/activity of the active ingredient(s) when administered, and compatible with the desired mode of administration, may be utilized as the pharmaceutically acceptable carrier in accordance with the present disclosure. In addition, the active ingredient(s) may be mixed with excipients which are pharmaceutically acceptable and compatible with the active ingredient(s). Suitable excipients include, for example but not by way of limitation, water, saline, dextrose, glycerol, ethanol, and the like, or any combination thereof.

The term “adoptive cell therapy” or “ACT” refers to the transfer of cells into a patient. The cells may have originated from the patient or from another individual or from an inducible pluripotent stem cell (iPSC).

The terms “polypeptide” and “protein” are used interchangeably herein and refer to any molecule that includes at least two or more amino acids.

The term “cancer” refers to any physiological condition in mammals characterized by unregulated cell growth. Cancers described herein include solid tumors and hematological (blood) cancers. A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The term “enhance” refers to an increase or improvement in the function or activity of a protein or cell after administration or contacting with a combination described herein compared to the protein or cell prior to such administration or contact.

The terms “inhibition,” “inhibit,” “inhibiting” refer to a reduction in the activity, binding, or expression of a polypeptide or reduction or amelioration of a disease, disorder, or condition or a symptom thereof. Inhibiting as used here can include partially or totally blocking stimulation, decreasing, preventing, or delaying activation or binding, or inactivating, desensitizing, or down-regulating protein or enzyme activity or binding.

As used herein, “Current Good Manufacturing Practice” or “cGMP” refers to the Current Good Manufacturing Practice regulations enforced by the US Food and Drug Administration (FDA) or equivalent regulatory authorities in non-US countries. cGMP regulations provide for systems that assure proper design, monitoring, and control of manufacturing processes and facilities. Adherence to the cGMP regulations assures the identity, strength, quality, and purity of drug products by requiring that manufacturers of medications adequately control manufacturing operations. This includes establishing strong quality management systems, obtaining appropriate quality raw materials, establishing robust operating procedures, detecting and investigating product quality deviations, and maintaining reliable testing laboratories.

As used herein, the term “ex vivo expansion” or “expansion” refers to a method of growing a cell population in tissue culture that increases the number of cells in that population. Cells that have undergone ex vivo expansion are referred to as “expanded.”

As used herein, the term “fucosylation” refers to the treatment of a population of cells with an α1,3-fucosyltransferase and fucose donor under conditions that increase the ability of the cells to bind to a selectin or that increase the reactivity of the cells with an antibody known in the art to bind to sLeX including, but not limited to, the HECA-452 monoclonal antibody. Cells that have been treated with an α1,3-fucosyltransferase and fucose donor and then exhibit increased binding to selectins or to the HECA-452 monoclonal antibody or to another antibody specific for sLeX are referred to as being “fucosylated.” As used herein, “fucosylation” can also refer to the levels of sLeX present on a cell population.

The term “chimeric antigen receptor T-cell” or “CAR-T cell” refers to T cells that have been genetically engineered to produce an artificial T cell receptor for use in immunotherapy. CAR-T cell therapy is a form of ACT that involves harvesting circulating lymphocytes from the patient, a separate donor, or iPSC; genetically engineering the harvested lymphocytes; culturing and amplifying the genetically engineered cells in vitro; and infusing the cultured/amplified genetically engineered lymphocytes into one or more patients for treatment of a condition or disorder.

Turning now to the inventive concepts, the present disclosure relates to the use of compositions and methods designed to provide a more effective and safer Hematopoietic Stem Cell Transplant therapy for the treatment of diseases such as (but not limited to) blood-based cancers, non-cancerous blood disorders, and autoimmune diseases through improving post-transplant recovery times and minimizing life threatening side effects of the transplant. The present disclosure is intended for use with child and adult patients as well as any individual who is in need of a Stem Cell Transplant for (for example, but not by way of limitation) any type of cancer, for non-cancerous hematologic conditions, and for individuals suffering from autoimmune disorders. The present disclosure also relates to methods of producing the surface-modified stem cells, methods to enhance their effects, medicaments comprising a combination therapy, and kits to deliver the therapy to the patients in an efficient and safe manner.

In HSCT therapy, the longer it takes for the stem cells to engraft and produce neutrophils, platelets, and other hemopoietic cells, the higher the risk of mortal consequences as a result of toxic infections (bacterial, viral, or fungal), uncontrolled internal bleeding, Host versus Graft Disease (HvGD), and graft failure. The inventors have discovered a means of significantly accelerating the recovery of hematopoietic cells post-transplant, through a combination of therapies using different mechanisms of action and an unexpected synergism that significantly improves the overall success rate of the HSCT therapy and results in a major reduction of patient deaths.

The inventors recognized that a key rate limiting step in the post-transplant hemopoietic recovery is the time it takes for the newly infused stem cells to escape the circulatory system and enter the body tissues and ultimately find their way to the bone marrow. Veinous blood velocities are typically from 1.5-7.0 cm/sec, depending on the diameter of the vessels. Consequently, the stem cells are moving very fast, and the process of squeezing between the endothelial cells to escape the veinous system (paracellular transmigration or diapedesis) requires that the movement of the stem cells is slowed and finally arrested in this fast-moving stream. Migration of hematopoietic stem cells through the blood, across the endothelial vasculature, and to the bone marrow (BM), requires active navigation; a process termed “homing” is an essential step in clinical stem cell transplantation. The process of stem cell capture, arrest, and transfer between adjacent endothelial cells and out of the circulatory system is facilitated by a mechanism involving surface receptors and binding sites on the stem cells and the endothelial cells.

The inventors have discovered that certain modifications to the stem cell surfaces (ex vivo fucosylation) can accelerate the rate and extent of the trafficking of the infused stem cells to the bone marrow by increasing their adhesion to the endothelial cells, thereby slowing them down and enabling the process of transmigration through the endothelial cell layer to the bone marrow micro-environment. However, when the stem cells reproduce by cell division, the surface fucosylation becomes diluted among the daughter cells, and the subsequent impacts of the fucosylation on the cells are typically lost within 48 hours, a time interval where cells home.

In addition, the inventors have discovered that the use of ex vivo fucosylated mononuclear cells increases the time to engraftment of stem cells as well as the rate of engraftment of stem cells. The mononuclear cell population includes stem cells as well as accessory cell types such as (but not limited to) T-cells (including cytotoxic, regulatory, and helper T-cells), NK cells, B-cells, dendritic cells, and/or macrophages, and the like. Ex vivo fucosylation of the mononuclear cell population results in fucosylation of not only the stem cells present therein but also fucosylation of these accessory cell types. The presence of fucosylated stem cells in combination with fucosylated accessory cell types provides synergistic effects upon subsequent administration of the fucosylated mononuclear cells to a patient. As shown in the Examples, engraftment of all infused cell types occurs must faster and at a much higher rate when compared to the infusion of control (unfucosylated mononuclear cells); in addition, a change in the composition of the engrafted cells is observed compared to control, enhancing the engraftment of desired cell types such as CD33⁺ and CD34⁺ cells. Therefore, the present disclosure provides a significant improvement in the time to recovery, increases tumor penetration and cytotoxicity, reduces the occurrence of relapse, accelerates the time to absolute neutrophil recovery, and reduces the occurrence of infections.

Once differentiated into immune cells (including, but not limited to, cytotoxic T-cells, regulatory T-cells, helper T-cells, NK-cells, B-cells, dendritic cells, and macrophages), there is a need to further expand those cells quickly in vivo to recover full immunological competence in the patient. Several compounds known as hematopoietic enhancers (HEs) have the ability to stimulate the production of neutrophils, erythrocytes, platelets, cytotoxic T-cells, regulatory T-cells, helper T-cells, NK-cells, B-cells, and/or dendritic cells and/or macrophages derived from the fucosylated stem cells and are a component of the present disclosure. Such compounds can be biologically active proteins/glycoproteins, growth factors, peptide mimics, or agonists, some of which mediate interactions between leukocytes and cytokines such as (but not limited to) IL2, IL12, and IL15. HEs are classified herein into at least four categories: (1) White Cell Enhancers (WCEs) such as, but not limited to, filgrastim (NEUPOGEN®, Amgen, Inc., Thousand Oaks, CA), PEGfilgrastim (NEULASTA®, Amgen, Inc.); (2) Red Cell Enhancers such as, but not limited to: erythropoietin and other erythropoiesis-stimulating agents (ESAs) (EPO; darbepoietin (dEPO); ARANESP®, Amgen, Inc.; EPOGEN2, Amgen, Inc.; EPREX®, Johnson and Johnson, New Brunswick, NI; and PROCRIT®, Johnson and Johnson), EPO-based constructs (EPO-Fc and methoxy polyethylene glycol-epoetin beta), continuous erythropoietin receptor activator (CERA), and EPO-mimetic agents and their constructs, CNTO-530 and peginesatide (OMONTYS®, Affymax, Inc., Palo Alto, CA; HEMATIDE®, Affymax, Inc.); and (3) Platelet Enhancers (PEs) such as, but not limited to: thrombopoetin (TPO), romiplostim (NPLATE®, Amgen, Inc.), eltrombopag, (PROMACTA®, Novartis Pharma AG, Basel, Switzerland; REVOLADE®, Glaxosmithkline LLC, Wilmington, DE), Avatrombopag (DOPTELET®, AkaRx, Inc. Durham, NC), and lusutrombopag (MULPLETA®, Shionogi & Co., LTD., Osaka, Japan).

Therefore, certain non-limiting embodiments of the present disclosure include the concomitant use of HEs with HSCT. The inventors discovered that when HEs are uniquely combined with fucosylated stem cells, the problem of the dilution of the fucosylation on the surface of the stem cells due to their expansion in vivo is overcome, and through a different mechanism of action, the neutrophils, eosinophils, platelets, cytotoxic T-cells, regulatory T-cells, helper T-cells, NK-cells, B-cells, and dendritic cells and macrophages expand at a greater rate and to a greater extent than they did with either step alone.

Certain non-limiting embodiments of the present disclosure include the concomitant use of one or more anti-cancer monoclonal antibodies with HSCT. The monoclonal antibodies may be used alone or in combination with one or more HEs.

Certain non-limiting embodiments of the present disclosure include the concomitant use of one or more growth factors (such as, but not limited to, IL2, IL7, IL8, IL10, IL12, IL15, IL23 (as well as agents based on any of these interleukins), G-CSF (i.e., filgrastim or Pegfilgrastim)) with HSCT. The growth factor(s) may be used alone or in combination with one or more HEs and/or one or more monoclonal antibodies.

Certain non-limiting embodiments of the present disclosure include the concomitant use of one or more anti-cancer vaccines with HSCT. The monoclonal antibodies may be used alone or in combination with one or more other products (such as, but not limited to, HEs, antibodies, and/or growth factors).

The inventors discovered that when fucosylated immune cells and other types of stem cells are co-infused with hematopoietic stem cells, fucosylation promotes the trafficking of these other cells to sites of inflammation/disease. These cells, such as (but not limited to) regulatory T cells, cytotoxic T-cells, T-helper cells, dendritic cells, macrophages, NK cells, and Gamma-Delta cells, express ‘growth factors’ such as (but not limited to) IL2 in sites of infection/inflammation which expands the immune cell population in those sites, thereby helping reduce the severity of infection, inflammation, and graft versus host disease, as well as slowing/halting the progression of the disease.

The present disclosure provides for compositions, methods, and protocols to significantly accelerate and increase the extent of the recovery of a patient's immune system during HSCT therapy for the treatment of hematopoietic cancers such as, but not limited to leukemia, lymphoma, and multiple myeloma including Acute myeloid (or myelogenous) leukemia (AML), Chronic myeloid (or myelogenous) leukemia (CML), Acute lymphocytic (or lymphoblastic) leukemia (ALL), Chronic lymphocytic leukemia (CLL), Hodgkin lymphoma, non-Hodgkin lymphoma (NHL), Light Chain Myeloma, Non-secretory Myeloma, Solitary Plasmacytoma, Extramedullary Plasmacytoma, Monoclonal Gammopathy of Undetermined Significance (MGUS), Smoldering Multiple Myeloma (SMM), Immunoglobulin D (IgD) Myeloma, and Immunoglobulin E (IgE) Myeloma.

The present disclosure can also be used in the treatment of other cancers including, but not limited to cancer of the prostate and skin, ovarian cancer, cancers of non-lymphoid parenchymal organs including the heart, placenta, skeletal muscle and lung, neuroblastoma or Ewing sarcoma, breast cancer, cancers of the head and neck including various lymphomas, such as mantle cell lymphoma, Non-Hodgkin B cell lymphoma, PTCL, adenoma, squamous cell carcinoma, laryngeal carcinoma, salivary carcinoma, thymomas and thymic carcinoma, leukemia, cancers of the retina, cancers of the esophagus, multiple myeloma, melanoma, colorectal cancer, lung cancer, cervical cancer, endometrium carcinoma, gallbladder cancer, liver cancer, thyroid follicular cancer, gastric cancer, non-small cell lung carcinoma, glioma, urothelial cancer, bladder cancer, prostate cancer, renal cell cancer, infiltrating ductal carcinoma, and glioblastoma multiform.

The present disclosure can also be used in the treatment of other non-cancerous autoimmune, metabolic, hematological, and non-hematological diseases including, but not limited to, sickle cell disease, thalassemia, inborn errors of metabolism, autoimmune diseases, and genetic diseases.

The present disclosure involves fucosylated stem cells. The stem cells can be sourced from human tissues including, but not limited to, fetal tissues including cord blood, infant or child tissue, marrow, adult blood and other tissues, and from differentiated somatic cells after they have been genetically reprogrammed as induced pluripotent stem cells (iPSCs) prior to ex vivo fucosylation. Specific stem cells in the present disclosure are prepared from peripheral blood (PBSCs), cord blood (CBSCs), mesenchmyal tissues (MSCs), or from somatic tissue that has been genetically reprogrammed as induced pluripotent stem cells (iPSCs). The stem cells of the present disclosure can be from any human source. In a particular (but non-limiting) embodiment of the present disclosure, the stem cells are harvested from the same person who is in need of the HSCT (i.e., the patient) and is referred to as an autologous transplant. If the stem cells are harvested from anyone other than the recipient, it is referred to an allogeneic transplant. The present disclosure can be practiced with both autologous and allogenic transplant procedures.

In one non-limiting embodiment of the present disclosure, fucosylated Mono Nucleated Cells (MNCs) or Total Nucleated Cells (TNCs) are included with fucosylated stem cells as part of the cell population that is infused into patients. If these cells are expanded ex vivo to increase their numbers prior to infusion into the patient, the fucosylation takes place before or after the expansion. In another non-limiting embodiment, specific cells such as, but not limited to, cytotoxic T-cells, regulatory T-cells, helper T-cells, NK-cells, B-cells, dendritic cells, and macrophages are fucosylated and given to the patients together with fucosylated stem cells. These cells when infused with the stem cells may directly or indirectly lead to a growth/expansion of immune cells in-vivo. In another non-limiting embodiment, the T-cells, NK cells, macrophages, etc. can be genetically modified by the insertion of a gene for Chimeric Antigen Receptor so that the T-cells can recognize a specific protein on the surface of diseased/cancer cells and thereby modify a patient's own immune cells (now CAR-immune cells) to recognize and attack cancer/diseased cells. The CAR gene is inserted into a viral vector such as, but not limited to, lentivirus or a retrovirus, and the vector is then used to infect the target immune cells. In other non-limiting embodiments of the present disclosure, immune-cell genetic modifications can include: the expression of a new immune-cell receptor (such as, but not limited to, a T-cell receptor (TCR)) that recognizes a specific tumor antigen; use of gene editing techniques such as CRISPR-Cas9 can be used to edit the genes in immune cells to enhance their anti-tumor activity by knocking out genes that inhibit immune-cell activity, or overexpressing genes that promote immune-cell activation and production of cytokines such as IL-2 or IL-12 that will enhance their ability to kill tumor cells and stimulate other immune cells; introduction of tumor-specific antigens to enhance their specificity for cancer cells; and introduction of suicide genes so that immune-cells can be eliminated if they cause harmful side effects. In the case of all genetic modifications of immune-cells, they shall take place prior to any ex vivo cell expansion while the fucosylation takes place after the ex vivo cell expansion.

Prior to the infusion of the stem cells into the patient, the stem cells and MNCs, which includes a diversity of cells such as, but not limited to MSCs, Cytotoxic T-cells, Regulatory T-cells, and NK cells, are fucosylated together by contact with a fucosyltransferase (FUT) and GDP-fucose for any time period and at any temperature that results in fucosylation of both cell types. Non-limiting examples of time periods include about 30 seconds, about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, or longer, as well as a range of time formed from two of the above values (e.g., a range of from about 5 to about 120 minutes, a range of from about 10 to about 90 minutes, a range of from about 20 to about 30 minutes, etc.). Non-limiting examples of temperatures that may be utilized in accordance with the present disclosure include about 10 °C, about 15 °C, about 20 °C, about 25 °C, about 30 °C, about 35 °C, about 40 °C, or higher, as well as a range formed from two of the above values (e.g., a range of from about 10 °C to about 40 °C, a range of from about 15 °C to about 35 °C, etc.). In a particular (but non-limiting) embodiment, the time for fucosylation is about 30 min and the temperature is about 37 °C.

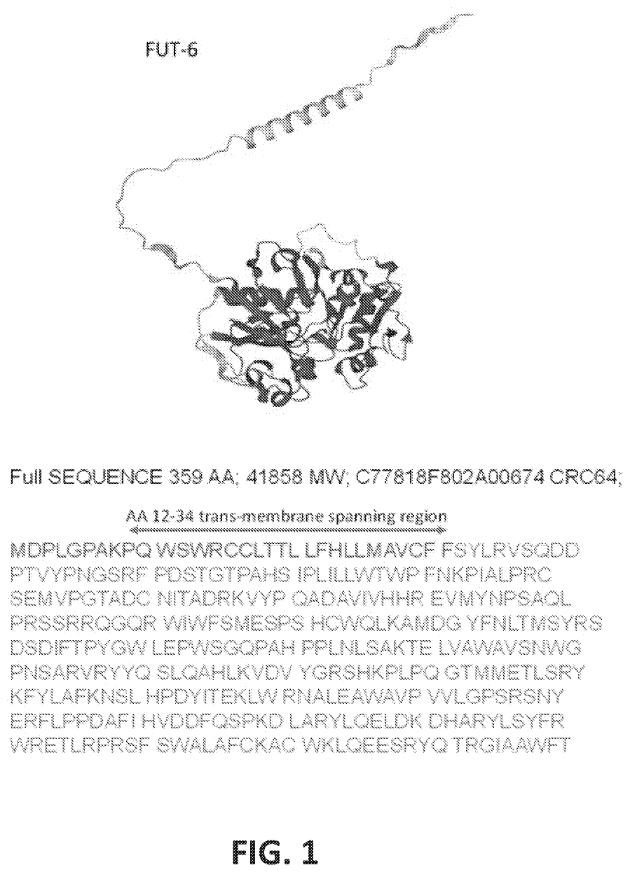

In a particular (but non-limiting) embodiment, the FUT is selected from, but not limited to, human gene FUT1, FUT2, FUT3, FUT4, FUT5, FUT6, FUT7, FUT8, FUT9, FUT10, and FUT11. In a particular (but non-limiting) embodiment, the FUT is FUT6 or FUT7. In one non-limiting embodiment of the present disclosure, the FUT is a recombinant product of the FUT gene that is produced in a mammalian, insect, bacterial, yeast, or fungal expression system and purified prior to contact with the stem cells and GDP fucose. In a particular (but non-limiting) embodiment, the membrane anchoring region of the FUT gene (FIG. 1) is removed in a way that makes the FUT enzyme soluble. In another non-limiting embodiment, the cloned gene also comprises a HIS-tag to aid in purification.

In one non-limiting embodiment of the present disclosure, other immune cells including, but to limited to cytotoxic T-cells, regulatory T-cells, helper T-cells, genetically modified T-cells, NK-cells, B-cells, dendritic cells, and macrophages may be fucosylated together and used in the transplant process. In another non-limiting embodiment of the present disclosure, HEs that are proteins or peptides can be fucosylated and used in the present disclosure. Non-limiting examples of HEs that can be fucosylated in accordance with the present disclosure include: (1) White Cell Enhancers such as (but not limited to) (WCEs) filgrastim (NEUPOGEN®, Amgen, Inc., Thousand Oaks, CA), PEGfilgrastim (NEULASTA®, Amgen, Inc., Thousand Oaks, CA); (2) Red Cell Enhancers (RCEs) such as (but not limited to) erythropoietin (EPO; Darbepoietin (dEPO); ARANESP®, Amgen, Inc.; EPOGEN®, Amgen, Inc.; EPREX®, Johnson and Johnson, New Brunswick, NJ; and PROCRIT®, Johnson and Johnson), EPO-based constructs (EPO-Fc and methoxy polyethylene glycol-epoetin beta), continuous erythropoietin receptor activator (CERA), peginesatide, and EPO-mimetic agents and their constructs) (CNTO-530®, peginesatide (OMONTYS®, Affymax, Inc., Palo Alto, CA; HEMATIDE®, Affymax, Inc.); and (3) Platelet Enhancers (PEs) such as (but not limited to) Thrombopoetin (TPO), romiplostim (NPLATE®, Amgen, Inc.) and eltrombopag, (PROMACTA®, Novartis Pharma AG, Basel, Switzerland; REVOLADES, Glaxosmithkline LLC, Wilmington, DE) Avatrombopag (DOPTELET®, AkaRx, Inc. Durham, NC), lusutrombopag (MULPLETA®, Shionogi & Co., LTD., Osaka, Japan), and the like.

Certain non-limiting embodiments of the present disclosure include medicaments that include compositions for the treatment of cancer and other diseases comprising at least one type of fucosylated stem cell and at least one type of HE and/or at least one monoclonal antibody, growth factor, or other immunotherapeutic. In another non-limiting embodiment, the composition comprises fucosylated stem cells and therapeutic cell enhancers of hematopoiesis including, but not limited to cytotoxic T-cells, regulatory T-cells, helper T-cells, NK-cells, B-cells, dendritic cells, CD4 positive and CD8 positive Tcells, activated dendritic cells and natural killer cells, macrophages, and other circulating cells that may produce immune cell growth factors including, but not limited to IL2, IL12, and IL15. In a particular (but non-limiting) embodiment, the composition comprises fucosylated therapeutic cell enhancers of hematopoiesis. In another particular (but non-limiting) embodiment, the composition comprises fucosylated stem cells, fucosylated therapeutic cell enhancers of hematopoiesis, and one or more hematopoietic enhancers (PEs). In yet another particular (but non-limiting) embodiment, the composition comprises fucosylated stem cells, fucosylated therapeutic cell enhancers of hematopoiesis, and one or more fucosylated hematopoietic enhancers (PEs). One non-limiting embodiment of the medicament would be peripheral blood stem cells (PBSCs) fucosylated by contacting the PBSCs with a native FUT or recombinant FUT (rFUT) of the human FUT-6 or FUT-7 genes along with GDP-Fucose for about 30 minutes at about 37 °C. A particular (but non-limiting) embodiment of the medicament would be the addition of a WCE such as, but not limited to, filgrastim or Pegfilgrastim to the previous composition, where the addition is made prior to, contemporaneous with, or after the infusion of the fucosylated PBSCs into the patient. Another non-limiting embodiment would be the addition of a RCE such as, but not limited to, erythropoetin or Peginesatide to the previous composition where the addition is made prior to, contemporaneous with, or after the perfusion of the fucosylated PBSCs into the patient. Another particular (but non-limiting) embodiment would be the addition of a PE such as, but not limited to romiplostim to the previous composition where the addition is made prior to, contemporaneous with, or after the perfusion of the fucosylated PBSCs into the patient. Further non-limiting embodiments would include various combinations of WCEs, RCEs, and PEs with the fucosylated SCs. In yet another particular (but non-limiting) embodiment of the Medicament, the PBSCs are initially harvested from the patient ultimately receiving the HSCT treatment (an autologous transplant), and the rFUT is a soluble form of the FUT with the anchor of the original protein deleted in the clone.

Certain non-limiting embodiments of methods of the present disclosure include starting with a peripheral blood draw from the patient in need of the transplant, isolating the PBSCs, and expanding the PBSCs using known cell culture procedures. The PBSCs are harvested, washed, and then fucosylated by contact with a native FUT or rFUT (FUT-6 or FUT-7) and GDP-Fucose. In one non-limiting embodiment, the fucosylation can be done in a cell culture medium enriched with fucose at from about 1% to about 5% by weight. After 30 minutes of fucosylation at 37 °C, the PBSCs are washed again to remove residual FUT/rFUT. At this time, the fucosylated PBSCs can be infused directly into the patient in need of the transplant. Alternatively, cryopreservatives can be added (as described by in US Patent Application Publication No. US2017/0121673, incorporated herein by reference in its entirety), and the PBSCs can be stored frozen until time of use. In some non-limiting embodiments of the present disclosure, the same fucosylation procedure is applied to the proteinaceous cell enhancers.

Prior to the infusion of the fucosylated PBSCs, the patient will undergo myeloablative chemotherapy for from 1-2 weeks using one of more immunosuppressants such as, but not limited to, anti-infective agents, Cyclophosphamide (CYTOXAN®, Ingenus Pharmaceuticals, Orlando, FL); Biologics such as but not limited to adalimumab (HUMIRA®, Abbvie Biotechnology LTD, Hamilton, Bermuda) and infliximab (REMICADE®, Janssen Biotech, Inc., Horsham, PA); Calcineurin inhibitors such as, but not limited to tacrolimus (Envarsus XR®, Veloxis Pharmacetuicals, Inc., Cary, NC; or PROTOPIC®, Leo Pharma A/S, Bellerup, Denmark) and cyclosporine (GENGRAF®, Abbvie Inc., North Chicago, IL; NEORAL®, Novartis AG; or SANDIMMUNE®, Novartis AG); Interleukin inhibitors such as, but not limited to, anakinra, canakinumab, rilonacept, sarilumab, tocilizumab, and siltuximab; Steroids and Corticosteroids such as, but not limited to Prednisone, methylprednisolone, dexamethasone, Colchicine, Hydroxychloroquine (PLAQUENIL®, Concordia Pharmaceuticals Inc., Luxembourg), Sulfasalazine, Dapsone, Methotrexate, Mycophenolate Mofetil (CELLCEPT®, Hoffman-La Roche Inc., South San Francisco, CA; MYFORTIC®, Novartis AG), Azathioprine (IMURAN®, Sebela Ireland Limited, Dublin, Ireland), Tacrolimus (PROGRAF®, Astellas Pharma US, Inc., Northbrook, IL), Sirolimus/Rapamycin (RAPAMUNE®, Wyeth LLC, New York, NY), Astagraf, Envarsus, Elidel, Pimecrolimus, Cequa, Protopic, and Restasis; TNFa inhibitors including, but not limited to Tocilizumab (Actemra), Tofacitinib Citrate (XELJANZ®, Pfizer Inc., New York, NY), Ustekinumab (STELARA®, Johnson & Johnson), Voclosporin (LUPKYNIS®, Aurinia Pharmaceuticals Inc., Victoria, BC, Canada), Adalimumab (HUMIRA®, Abbvie Biotechnology LTD), Certolizumab pegol (CIMZIA®, UCB Pharma, Brussels, Belgium), Etanercept (ENBREL®, Immunex Corp, Thousand Oaks, CA), Etanercept-szzs (Ereizi), Golimumab (SIMPONI® and SIMPONI ARIA®, Johnson & Johnson), Infliximab (REMICADE®, Janssen Biotech, Inc.), Infliximab-abda (Renflexis), Adalimumab (HUMIRA®, Abbvie Biotechnology LTD), Adalimumab-adbm (CYLTEZO®, Boehringer Ingelheim International GmbH, Germany), Adalimumab-adaz (HYRIMOZ®, Novartis AG), and Adalimumab-atto (AMGEVITA®, Amgen, Inc.). Upon completion of the myeloablation period, the patient is infused with the medicament comprising the fucosylated PBSCs cell one or more cell enhancers selected from a group of WCEs, RCEs and PEs and/or one of more fucosylated therapeutic cell types that facilitate hematopoiesis such as, but not limited to, cytotoxic T-cells, regulatory T-cells, helper T-cells, NK-cells, B-cells, dendritic cells, and macrophages. Each of the components of the medicament can be provided prior to, contemporaneously with, or following the infusion of the PBSCs. In a particular (but non-limiting) embodiment, all components are provided simultaneously in the same intravenous infusion bag with the PBSCs. In one non-limiting embodiment of the present disclosure, the protein-based cell enhancers are also fucosylated using the same procedure as for fucosylation of the proteins on the surface of the stem cells.

Certain non-limiting embodiments of the present disclosure are directed to a kit that provides any of the components described herein. In a particular (but non-limiting) embodiment, the kit provides each of the individual HE components to be provided to the patient at the time of the infusion of the PBSCs. This ensures strict maintenance to the HSCT protocol.

Additional non-limiting embodiments of the present disclosure are described in the Examples and Non-Limiting Illustrative Embodiments sections below.

### EXAMPLES

Examples are provided hereinbelow. However, the present disclosure is to be understood to not be limited in its application to the specific experimentation, results, and laboratory procedures disclosed herein after. Rather, the Examples are simply provided as one of various embodiments and are meant to be exemplary, not exhaustive.

### Example 1: Preparation of Stem Cells

Peripheral blood stem cells (PBSCs) are collected from whole blood usually from the patient to be treated by HSCT as they will be the best genetic match. Before PBSC or bone marrow collection, the donor (either the patient or a healthy donor) receives an HE such as filgrastim to stimulate the production of stem cells in the bone marrow and move them into the bloodstream. This process is called mobilization. The donor's blood is collected through a vein in their arm and is passed through a cell separator to separate the stem cells from the other blood cells, such as red blood cells and white blood cells. The remaining blood is returned to the donor through a vein in their other arm. The stem cells are collected in a sterile bag and transported to the laboratory for processing. In the laboratory, the stem cells are processed to remove any remaining red blood cells or plasma. The stem cells are then frozen and stored until they are ready for transplant. In one non-limiting embodiment of the present disclosure, the stem cells can be expanded in cell culture before freezing. The collected PBSCs are tested for their viability, sterility, and cell count to ensure that they are suitable for transplantation. When the PBSCs are needed for transplant, they are thawed and prepared for infusion into the patient. PBSC collection is a less invasive and less painful procedure than bone marrow collection, and it can often provide a larger number of stem cells for transplantation.

When using cord blood to collect stem cells (CBSCs), the cord blood is collected immediately after the birth of the baby, either from the umbilical cord or the placenta, which are normally discarded after birth. The donation process does not harm the baby or the mother. The collected cord blood is transported to the laboratory (or cord blood bank), where it undergoes processing to extract the stem cells. The processing involves separating the stem cells from the other components of the cord blood, such as red blood cells and plasma. The remaining cord blood is stored for future testing or research. The collected cord CBSCs are tested for their viability, sterility, and cell count to ensure that they are suitable for transplantation. The cord blood stem cells are frozen and stored in a liquid nitrogen freezer until they are needed for transplantation. In one non-limiting embodiment of the present disclosure, the stem cells can be expanded in cell culture before freezing. In another non-limiting embodiment, the stem cells can be fucosylated prior to freezing. When a patient is ready to receive a CBSC transplant, the cord blood stem cells are thawed and prepared for infusion into the patient. Because cord blood contains a smaller number of stem cells compared to bone marrow or peripheral blood, it may be necessary to combine cord blood from two different donors to achieve a sufficient number of stem cells for transplantation.

The collection of mesenchymal stem cells (MSCs) can vary depending on the source of the MSCs. MSCs can be derived from various tissues, including bone marrow, adipose tissue, and umbilical cord and placental tissue. Collecting MSCs from bone marrow first requires that the bone marrow is harvested from the iliac crest (hip bone) of the donor using a needle and syringe. The donor is given local anesthesia to minimize discomfort. The bone marrow is processed to extract the MSCs. This process involves separating the MSCs from other components of the bone marrow, such as red blood cells and white blood cells and is done using density gradient centrifugation, in which the bone marrow is layered on top of a density gradient solution and centrifuged, causing the different cell types to separate based on their density. The extracted MSCs are expanded in cell culture to increase their number by placing the cells in a specialized culture medium that contains growth factors and other nutrients that promote cell growth and proliferation. The MSCs are tested for their viability, sterility, and cell count to ensure that they are suitable for transplantation. The expanded MSCs are frozen and stored in a liquid nitrogen freezer until they are needed for transplantation. When collecting MSCs from adipose tissue, the tissue is harvested using liposuction and processed to extract the MSCs. When collecting MSCs from umbilical cord or placental tissue, the tissue is collected after the birth of the baby and processed to extract the MSCs. The extracted MSCs are then expanded and cryopreserved using similar techniques as described above.

### Example 2: Preparation and Testing of Fucosyltransferase

A fucosyltransferase to be used in the present disclosure is prepared by starting with the full sequence of the FUT-6 or FUT-7 or other FUT-relevant genes isolated from a DNA library or genomic DNA using PCR amplification. The full genomic version of the gene contains a hydrophobic domain that anchors it in the membrane where it is normally located (see FIG. 1). This hydrophobic domain is removed, and the PCR product is purified and cloned into a suitable vector, such as a plasmid, using restriction enzymes and ligase. The resulting vector is used to transform a suitable bacterial, yeast, or fungal) host cell, such as Escherichia coli, Pichia pastoris, or Aspergillus oryzae for amplification and purification of the FUT-6 gene product. Optionally, the PCR product missing the hydrophobic tether is expressed in an insect cell expression system using a baculovirus transformation vector wherein the amino acid residues 1-67 of the FUT 6 gene, which comprise the signal peptide transmembrane region, are replaced by the sequence of the signal peptide of gp67 of the baculovirus for efficient secretion. Optionally, the PCR product missing the hydrophobic tether is expressed in an appropriate mammalian cell line. Chinese Hamster Ovary (CHO) cells are used for expressing the FUT-6 gene construct because they are easy to culture and have a high capacity for protein rFUT production. There are different CHO cell lines available, and the selection of the appropriate line depends on the desired expression level, post-translational modification requirements, and other factors. The modified FUT-6 gene is cloned into an appropriate vector for expression in CHO cells, such as one that comprises the cytomegalovirus (CMV) promoter, and a selection marker to allow for selection of cells that have taken up the vector. The foreign gene-containing vector is introduced into the CHO cells using Lipofectamine as a transfection reagent. Different methods can be used for transfection, including electroporation, calcium phosphate transfection, and viral transduction. The transfected cells are then selected using the appropriate selection marker. The transfected cells are screened for high-level expression of the foreign gene using techniques such as Western blotting or ELISA. High-producing cell clones are selected and further characterized for protein expression level, stability, and other factors. The high-producing cell clones are grown in large-scale cell culture, and the recombinant Fucosyltransferase (rFT) is secreted into the culture medium and purified using affinity chromatography or ion-exchange chromatography.

The final purified rFUT is then tested for enzymatic activity and its ability to fucosylate stem cells. Fucosylation of stem cells is measured using flow cytometry, which involves labelling cells with antibodies that recognize fucosylated proteins on the cell surface and passing the labelled cells through a flow cytometer, which detects and quantifies the fluorescent signal emitted by the labeled cells. The enzyme is considered usable in the present disclosure if it can produce a 4-fold or better signal in the stem cells vs. control when the enzyme is added along with its substrate GDP-Fucose in the cell culture medium at 37 °C within 30 minutes.

### Example 3: Fucosylation of Stem Cells

Fucosylation is the process of adding fucose sugars to glycoproteins. The materials required for this step include the stem cells of interest, the rFUT enzyme of Example 2, GDP-fucose or other fucose donor substrates, Dulbecco's phosphate-buffered saline (DPBS) or other cell culture medium, Cell culture plates or dishes, Sterile pipettes and tips, and an Incubator. Stem cells are cultured in DPBS or other cell culture medium until they reach around 70-80% confluency. The fucose donor substrate (GDP-fucose) is prepared by dissolving it in DPBS or other cell culture medium according to the manufacturer's instructions. The rFUT enzyme of Example 2 is added to the fucose donor substrate solution and mixed well. The culture medium of the stem cells is replaced with the fucosyltransferase-fucose substrate mixture and the stem cells are allowed to incubate at 37 °C in a humidified incubator for 30-60 minutes. After incubation, the fucose substrate mixture is removed and the fucosylated stem cells are washed with DPBS cell culture medium to remove any unbound enzyme or substrate and the fucosylated stem cells are ready to be infused into a patient.

### Example 4a: Preparation of Non-therapeutic Hematopoietic Cell Enhancers

Hematopoietic Cell Enhancers (HCEs) are generally glycoproteins or glycoprotein fragments and may be used directly in medicaments comprising fucosylated stem cells or they may be fucosylated as well using the procedure of Example 3 prior to making the medicaments of the present disclosure. Medicaments are made by adding the HCEs to the fucosylated stem cells of Example 3. In separate containers Solutions of the White Cell Enhancer (WCE) filgrastim (NEUPOGEN®, Amgen, Inc., Thousand Oaks, CA) at 5 μg/ml, the red cell enhancer (RCE) erythropoietin (EPOGEN®, Amgen, Inc.) at 20-50 units/ml, and the platelet enhancer (PE) romiplostim (NPLATE®, Amgen, Inc.) at 0.5-1.5 μg/ml are made up in separate containers at concentrations in PBS. Seven unique medicaments are prepared as a kit (Table 1) representing different combinations of HEs with fucosylated stem cells. This kit can be used for preclinical testing on the impacts of various HEs with fucosylated stem cells on different types of animal models of cancer prior to use in the clinic.

TABLE 1

| | | White Cell | Red Cell | Platelet |
|---|---|---|---|---|
| | Stem Cells | Enhancer | Enhancer | Enhancer |
| Medicament 1 | √ | √ | | |
| Medicament 2 | √ | | √ | |
| Medicament 3 | √ | | | √ |
| Medicament 4 | √ | √ | √ | |
| Medicament 5 | √ | √ | | √ |
| Medicament 6 | √ | | √ | √ |
| Medicament 7 | √ | √ | √ | √ |

### Example 4b: Preparation of Therapeutic Cells as Enhancers of Hematopoiesis

A mixture of several therapeutic cell types including CD4 positive T-cells, CD8 positive T cells and innate immune cells such as activated dendritic cells (DCs), natural killer (NK) cells, and macrophages are combined with the stem cells of Example 1 and the mixture is fucosylated according to the procedure of Example 3. This novel mixture of fucosylated stem cells and fucosylated therapeutic immune cells are transferred to an IV delivery bag and infused into a patient in need of HSCT. This results in the acceleration of the expansion of all populations of immune cells and stem cells leading to improved patient outcomes.

### Example 5: Treatment of a Patient with a Hematopoietic Cancer Using PBSCs or BM Cells

A patient with a hematopoietic cancer such as leukemia, lymphoma, and multiple myeloma Lymphoma, or a blood disorder, autoimmune disease, metabolic disease, or genetic disease and in need of HSCT is identified. The goal of HSCT is to replace the patient's diseased or damaged bone marrow with healthy donor stem cells, which can help restore the immune system with the fastest recovery of new blood cells and the lowest rate of adverse events. Before stem cell transplantation, the patient undergoes a thorough medical evaluation, including blood tests, imaging scans, and a physical examination. The patient receives conditioning therapy, which involves high-dose tacrolimus chemotherapy and/or radiation to destroy any remaining cancer cells and to suppress the immune system to prevent rejection of the transplanted cells. Donor selection and evaluation is done to determine the best match for the patient. The donor may be the patient, a close family member, or an unrelated health donor. Stem cell mobilization is performed, where the donor is given growth factors such as filgrastim to increase the number of stem cells in their bloodstream. These stem cells are then collected from donor's peripheral blood as peripheral blood mononuclear cells (PBMCs) through a process called apheresis. After the PBMCs have been collected, peripheral blood stem cells (PBSCs) are separated using one of several stem cell isolation methods by virtue of their expression of the stem cell marker CD34. The stem cells are expanded and then fucosylated by contacting the purified PBSCs with an rFUT from Example 2 using the process of Example 3. The patient then receives the fucosylated stem cells through a central venous catheter, contemporaneously with HEs as part of the particular (but non-limiting) HE medicament of Example 4. The stem cells migrate to the bone marrow and begin to produce new blood cells. The patient receives the particular (but non-limiting) medicament of Example 4 daily until the hemopoietic cell numbers reach the target goal. The patient is closely monitored for signs of complications, such as infections, graft-versus-host disease (GVHD), and rejection of the transplant. The patient may require additional supportive care, such as blood transfusions, antibiotics, and antifungal medications. The patient is gradually weaned off immunosuppressive medications as their new immune system develops. Long-term follow-up is done to monitor the patient's health and to address any issues that may arise.

### Example 6: Treatment of a Patient with a Non-Cancerous Blood Disorder Using CBSCs

Treating a patient with sickle cell disease or thalassemia using stem cells involves Hematopoietic Stem Cell Transplantation (HSCT), which replaces the patient's defective blood-forming stem cells with healthy stem cells from a donor. Before the HSCT, the patient undergoes a series of tests to assess their overall health, including blood tests, imaging studies, and possibly a bone marrow biopsy. The patient's tissue type is also determined to identify a suitable donor. A donor with a matching tissue type is identified, either from a family member or from an unrelated donor registry. The donor undergoes a process called leukapheresis, which involves the collection of blood stem cells through a needle in the arm. The stem cells are separated from the donor's blood and prepared for transplantation. Prior to the HSCT, the patient receives a conditioning regimen, which involves high-dose chemotherapy and possibly radiation therapy. The goal of this regimen is to destroy any remaining defective blood-forming stem cells in the patient's bone marrow and suppress the patient's immune system to prevent rejection of the transplanted cells. The collected stem cells are expanded and then then fucosylated by contacting the purified PBSCs with an rFUT from Example 2 using the process of Example 3. The patient then receives the fucosylated stem cells through a central venous catheter, immediately followed by the particular (but non-limiting) HE medicament of Example 4. The patient continues receiving the particular (but non-limiting) medicament from Example 4 on a daily basis until the hemopoietic cell numbers reach the target goal. The transplanted stem cells will travel to the bone marrow and begin producing new blood cells, including red blood cells, white blood cells, and platelets. Post Transplantation, the patient is closely monitored for any signs of complications, such as infection or graft-versus-host disease (GVHD), which occurs when the donor cells attack the patient's own tissues. The patient may need to stay in the hospital for several weeks or months after the transplant. Once the patient's blood counts have recovered and there are no signs of complications, the patient will cease taking the medicaments of Example 4 and is discharged from the hospital. The patient continues to receive regular follow-up care to monitor for any signs of transplant-related complications and to manage any long-term effects of the transplant, such as GVHD or delayed growth and development. The success of HSCT in treating sickle cell disease or thalassemia depends on several factors, Including the patient's age and overall health, the severity of their disease, and the availability of a suitable donor.

### Example 7: Treatment of a Patient with a Solid Tumor Using Stem Cells

Cord blood stem cell transplant (CBSCT), peripheral blood stem cell transplant (PBSCT), or bone marrow transplant (BMT) can be used as a treatment option for patients with solid tumors such as neuroblastoma or Ewing sarcoma. High-risk neuroblastoma is a solid tumor that arises from developing nerve cells and is often seen in children. In high-risk neuroblastoma, the cancer has spread extensively, and the prognosis is poor. CBSCT is used as part of a treatment plan that includes chemotherapy and radiation. The goal of the transplant is to replace the patient's damaged bone marrow with healthy stem cells from cord blood, which can produce new blood cells and restore the immune system. The transplanted cells also have an anti-tumor effect, as they can recognize and attack the cancer cells. The process starts with identifying a patient with high-risk neuroblastoma and in need of HSCT. Before the CBSCT, the patient undergoes a series of tests to assess their overall health, including blood tests, imaging studies, and possibly a bone marrow biopsy. Prior to the CBSCT, the patient receives a conditioning regimen, which involves high-dose chemotherapy and possibly radiation therapy. The goal of this regimen is to destroy any remaining cancer cells and suppress the patient's immune system to prevent rejection of the transplanted cells. Cord blood units are collected from a donor or blood bank. The cord blood is tested for compatibility with the patient's tissue type and undergoes processing to remove red blood cells and plasma. The stem cells along with the mixture of other therapeutic cells from the cord blood are collectively expanded and fucosylated by contact with an rFUT from Example 2 using the process of Example 3. The patient then receives the fucosylated stem cells through a central venous catheter, followed the particular (but non-limiting) HE medicament of Example 4 contemporaneously. The patient receives the cord blood stem cells through a central venous catheter along with the particular (but non-limiting) HE combination from Example 4 provided extemporaneously with the stem cells. The patient continues to get daily doses of the medicament of Example 4 until the hemopoietic cell numbers reach their target goal. The transplanted stem cells travel to the bone marrow and produce new blood cells, including red blood cells, white blood cells, and platelets. The patient is closely monitored for any signs of complications, such as infection or graft-versus-host disease (GVHD), which occurs when the donor cells attack the patient's own tissues. The patient may need to stay in the hospital for several weeks or months after the transplant. Once the patient's blood counts have recovered and there are no signs of complications, the patient is discharged from the hospital. The patient continues to receive regular follow-up care to monitor for any signs of cancer recurrence and to manage any long-term effects of the transplant, such as GVHD or delayed growth and development.

### Example 8: Treatment of a Cancer Patient with Fucosylated Cells and Monoclonal Antibodies

A patient with a cancer in need of a stem cell transplant (such as, but not limited to, a HSCT) is identified. The goal of HSCT is to replace the patient's diseased or damaged bone marrow with healthy donor stem cells, which can help restore the immune system with the fastest recovery of new blood cells and the lowest rate of adverse events. Before stem cell transplantation, the patient undergoes a thorough medical evaluation, including blood tests, imaging scans, and a physical examination. The patient receives conditioning therapy, which involves high-dose tacrolimus chemotherapy and/or radiation to destroy any remaining cancer cells and to suppress the immune system to prevent rejection of the transplanted cells. Donor selection and evaluation is done to determine the best match for the patient. The donor may be the patient, a close family member, or an unrelated health donor. Stem cell mobilization is performed, where the donor is given growth factors such as filgrastim to increase the number of stem cells in their bloodstream. These stem cells are then collected from donor's peripheral blood as peripheral blood mononuclear cells (PBMCs) through a process called apheresis. After the PBMCs have been collected, peripheral blood stem cells (PBSCs) are separated using one of several stem cell isolation methods by virtue of their expression of the stem cell marker CD34. The stem cells are expanded and then fucosylated by contacting the purified PBSCs with an rFUT from Example 2 using the process of Example 3. The patient then receives the fucosylated stem cells through a central venous catheter, contemporaneously with HEs as part of the particular (but non-limiting) HE medicament of Example 4. The stem cells migrate to the bone marrow and begin to produce new blood cells. The patient receives the hematopoietic enhancer daily until the hemopoietic cell numbers reach the target goal. The patient also receives at least one monoclonal antibody one or more times during this treatment cycle. The at least one monoclonal antibody is selected from those listed in Table 2.

The patient is closely monitored for signs of complications, such as (but not limited to) infections, graft-versus-host disease (GVHD), and rejection of the transplant. The patient may require additional supportive care, such as blood transfusions, antibiotics, and antifungal medications. The patient is gradually weaned off immunosuppressive medications as their new immune system develops. Long-term follow-up is done to monitor the patient's health and to address any issues that may arise.

TABLE 2

| Monoclonal | | | First Indication |
|---|---|---|---|
| Antibody | Brand Name | Target; Format | Approved/Reviewed |
| Elranatamab | Elrexfio | BCMA, CD3; Humanized IgG2 | Multiple myeloma |
| Talquetamab | TALVEY | G protein-coupled receptor 5D, | Multiple myeloma |
| | | CD3; Humanized IgG4 bispecific | |
| Epcoritamab | EPKINLY ™ | CD20, CD3; Bispecific | Diffuse large B cell |
| | | humanized IgG1 | lymphoma |
| Glofitamab | Columvi ® | CD20, CD3e; Bispecific 2 + 1 IgG1 | Diffuse large B-cell |
| | | CrossMab | lymphoma |
| Tislelizumab | TEVIMBRA | PD-1; Humanized IgG4 | Esophageal squamous cell |
| | | | carcinoma |
| Toripalimab | LOQTORZI, | PD-1; Humanized IgG4 | Nasopharyneal carcinoma, |
| | Tuoyi | | esophageal squamous cell |
| | | | carcinoma |
| Retifanlimab | Zynyz | PD-1; Humanized IgG4 | Merkel cell carcinoma |
| Ublituximab | BRIUMVI | CD20; Chimeric IgG1 | Multiple sclerosis |
| Mirvetuximab | ELAHERE | Folate receptor alpha; | Ovarian cancer |
| soravtansine | | Humanized IgG1 ADC | |
| Nirsevimab | Beyfortus | RSV; Human IgG1 | RSV infection |
| Tremelimumab | Imjudo | CTLA-4; Human IgG2A | Antineoplastic; liver cancer |
| Teclistamab | TECVAYLI | BCMA, CD3; Humanized | Multiple myeloma |
| | | bispecific IgG4 | |
| Mosunetuzumab | Lunsumio | CD20, CD3; Humanized | Follicular lymphoma |
| | | bispecific IgG1 | |
| Relatlimab | Opdualag | LAG-3; Human IgG4 | Melanoma |
| | (relatlimab + | | |
| | nivolumab | | |
| | combo) | | |
| Tebentafusp | KIMMTRAK | gp100, CD3; Bispecific | Metastatic uveal melanoma |
| | | immunoconjugate (TCR-scFv) | |
| Tisotumab vedotin | TIVDAK | Tissue factor; Human IgG1 ADC | Cervical cancer |
| Amivantamab | Rybrevant | EGFR, cMET; Human bispecific | NSCLC w/EGFR exon 20 |
| | | IgG1 | insertion mutations |
| Loncastuximab | Zynlonta | CD19; Humanized IgG1 ADC | Diffuse large B-cell |
| tesirine | | | lymphoma |
| Dostarlimab | Jemperli | PD-1; Humanized IgG4 | Endometrial cancer |
| Margetuximab | MARGENZA | HER2; Chimeric IgG1 | HER2+ breast cancer |
| Naxitamab | DANYELZA | GD2; Humanized IgG1 | High-risk neuroblastoma and |
| | | | refractory osteomedullary |
| | | | disease |
| Belantamab | BLENREP | BCMA; Humanized IgG1 ADC | Multiple myeloma |
| mafodotin | | | |
| Tafasitamab | Monjuvi, | CD19; Humanized IgG1 | Diffuse large B-cell |
| | Minjuvi | | lymphoma |
| Sacituzumab | Trodelvy | TROP-2; Humanized IgG1 ADC | Triple-neg. breast cancer |
| govitecan | | | |
| Isatuximab | Sarclisa | CD38; Chimeric IgG1 | Multiple myeloma |
| [fam]-trastuzumab | Enhertu | HER2; Humanized IgG1 ADC | HER2+ breast cancer |
| deruxtecan | | | |
| Enfortumab | Padcev | Nectin-4; Human IgG1 ADC | Urothelial cancer |
| vedotin | | | |
| Polatuzumab | Polivy | CD79b; Humanized IgG1 ADC | Diffuse large B-cell |
| vedotin | | | lymphoma |
| Cemiplimab | Libtayo | PD-1; Human IgG4 | Cutaneous squamous cell |
| | | | carcinoma |
| Moxetumomab | Lumoxiti | CD22; Murine IgG1 dsFv | Hairy cell leukemia |
| pasudotox | | immunotoxin | |
| Mogamulizumab | Poteligeo | CCR4; Humanized IgG1 | Cutaneous T cell lymphoma |
| Durvalumab | IMFINZI | PD-L1; Human IgG1 | Bladder cancer |
| Inotuzumab | BESPONSA | CD22; Humanized IgG4, ADC | Hematological malignancy |
| ozogamicin | | | |
| Avelumab | Bavencio | PD-L1; Human IgG1 | Merkel cell carcinoma |
| Atezolizumab | Tecentriq | PD-L1; Humanized IgG1 | Bladder cancer |
| Olaratumab | Lartruvo | PDGRFα; Human IgG1 | Soft tissue sarcoma |
| Daratumumab | Darzalex | CD38; Human IgG1 | Multiple myeloma |
| Elotuzumab | Empliciti | SLAMF7; Humanized IgG1 | Multiple myeloma |
| Necitumumab | Portrazza | EGFR; Human IgG1 | Non-small cell lung cancer |
| Dinutuximab | Qarziba; | GD2; Chimeric IgG1 | Neuroblastoma |
| | Unituxin | | |
| Nivolumab | Opdivo | PD1; Human IgG4 | Melanoma, non-small cell |
| | | | lung cancer |
| Blinatumomab | Blincyto | CD19, CD3; Murine bispecific | Acute lymphoblastic |
| | | tandem scFv | leukemia |
| Pembrolizumab | Keytruda | PD1; Humanized IgG4 | Melanoma |
| Ramucirumab | Cyramza | VEGFR2; Human IgG1 | Gastric cancer |
| Obinutuzumab | Gazyva | CD20; Humanized IgG1; | Chronic lymphocytic |
| | | Glycoengineered | leukemia |
| Ado-trastuzumab | Kadcyla | HER2; Humanized IgG1, ADC | Breast cancer |
| emtansine | | | |
| Pertuzumab | Perjeta | HER2; Humanized IgG1 | Breast Cancer |
| Brentuximab | Adcetris | CD30; Chimeric IgG1, ADC | Hodgkin lymphoma, |
| vedotin | | | systemic anaplastic large cell |
| | | | lymphoma |
| Ipilimumab | Yervoy | CTLA-4; Human IgG1 | Metastatic melanoma |
| Ofatumumab | Arzerra | CD20; Human IgG1 | Chronic lymphocytic |
| | | | leukemia |
| Panitumumab | Vectibix | EGFR; Human IgG2 | Colorectal cancer |
| Bevacizumab | Avastin | VEGF; Humanized IgG1 | Colorectal cancer |
| Cetuximab | Erbitux | EGFR; Chimeric IgG1 | Colorectal cancer |
| Tositumomab-I131 | Bexxar | CD20; Murine IgG2a | Non-Hodgkin lymphoma |
| Ibritumomab | Zevalin | CD20; Murine IgG1 | Non-Hodgkin lymphoma |
| tiuxetan | | | |
| Alemtuzumab | MabCampath, | CD52; Humanized IgG1 | Chronic myeloid leukemia#; |
| | Campath-1H; | | multiple sclerosis |
| | Lemtrada | | |
| Gemtuzumab | Mylotarg | CD33; Humanized IgG4, ADC | Acute myeloid leukemia |
| ozogamicin | | | |
| Trastuzumab | Herceptin | HER2; Humanized IgG1 | Breast cancer |
| Rituximab | MabThera, | CD20; Chimeric IgG1 | Non-Hodgkin lymphoma |
| | Rituxan | | |
| Edrecolomab | Panorex | EpCAM; Murine IgG2a | Colorectal cancer |

### Example 9: Use of Fucosylated Stem Cells Enhances Engraftment Following HSCT

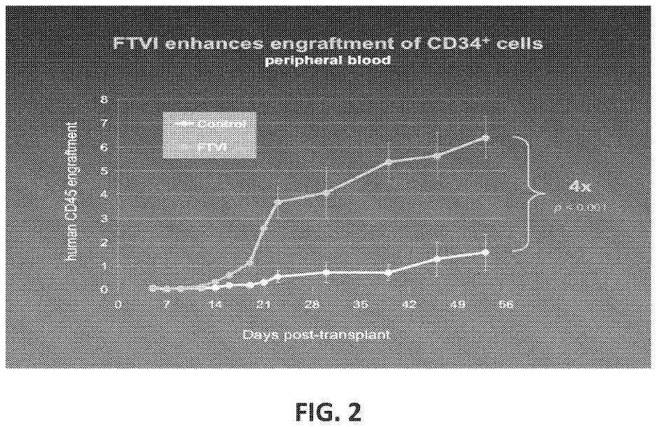

FIG. 2 illustrates that fucosylation enhances both the rate and amount of engraftment of hematopoietic stem cells after transplantation. This acceleration in engraftment will lead to faster reconstitution of the different cell types in blood and greatly reduce the risk of graft failure.

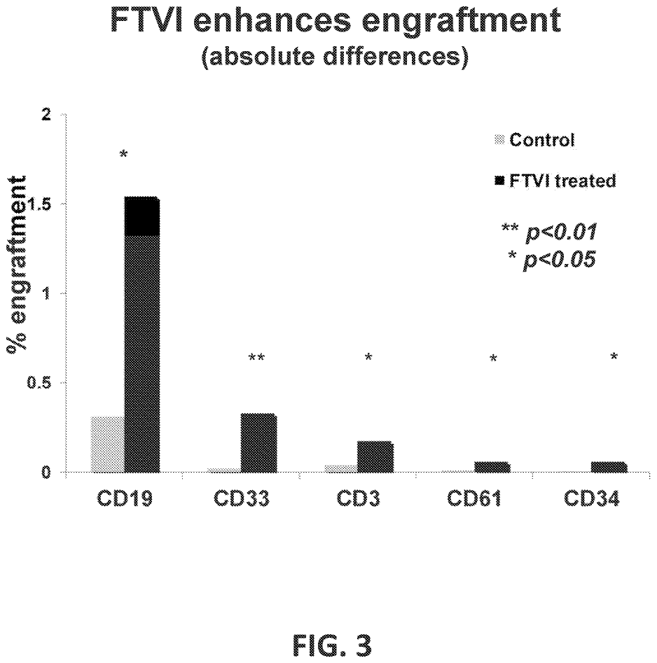

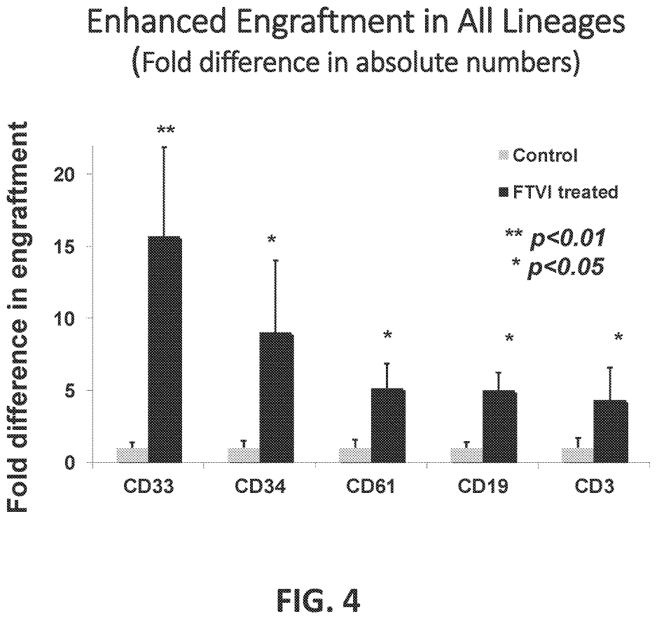

FIGS. 3-4 illustrates that ex vivo fucosylation of mononuclear cells prior to transplantation enhances the engraftment of all mononuclear cell lineages when compared to transplantation of non-fucosylated mononuclear cells; FIG. 3 illustrates the difference in overall percentage of engraftment, while FIG. 4 illustrates the fold difference in engraftment. The changes in cell composition post engraftment show a significant increase in all mononuclear cell types/lineages. For example, engraftment of CD19⁺ cells (i.e., B-lineage cells) following infusion with ex vivo fucosylated mononuclear cells was exponentially greater than that observed for the control (engraftment of CD19⁺ cells following infusion with mononuclear cells that were not ex vivo fucosylated). Similar results were also obtained with CD33⁺ cells (i.e., cells of lymphoid lineage), CD3⁺ cells (i.e., T cells), CD61⁺ cells (i.e., megakaryocytes), and CD34⁺ cells (hematopoietic stem cells).

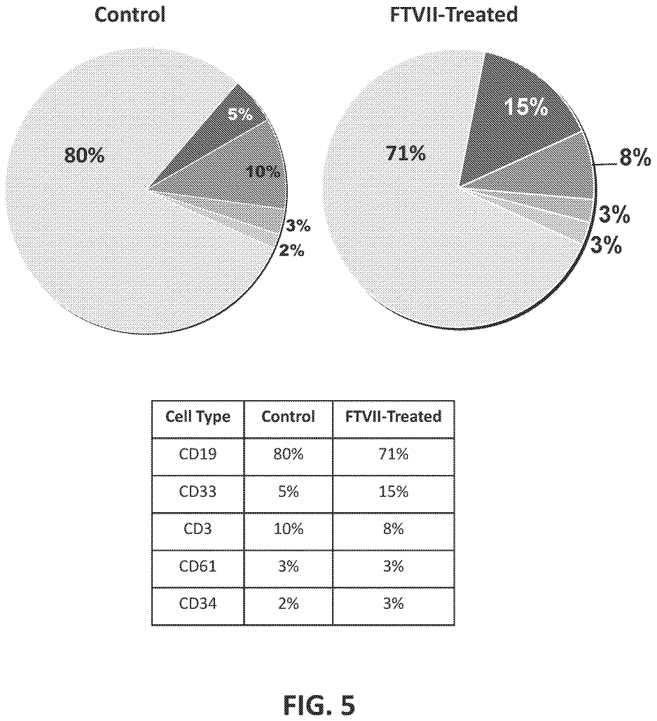

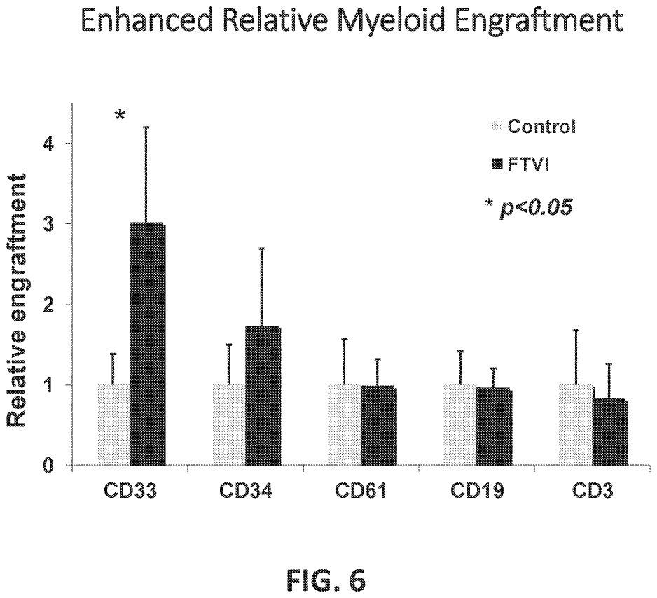

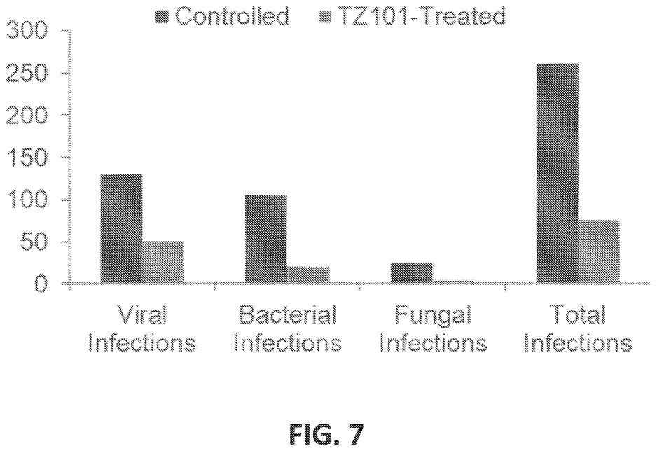

FIGS. 5-6 demonstrate that the phenotypes of engrafted cells actually change with the use of ex vivo fucosylation prior to implantation. In particular, FIG. 5 demonstrates that fucosylation enhances relative engraftment of the lymphoid lineage; the increase in engraftment of the lymphoid lineage ensures that more T cells can home to the tumor microenvironment, providing better infiltration into the tumor, increasing tumor cytotoxicity, and reducing the occurrence of relapse. In addition, FIG. 6 demonstrates that fucosylation enhances relative engraftment of the myeloid lineage; the increase in engraftment of the myeloid lineage ensures accelerated time to absolute neutrophil recovery and reduction in infections. This can be seen in FIG. 7, which demonstrates that fucosylation significantly reduced the number of episodes of infection in patients after completion of HSCT, compared to historical controls. It should be noted that the fucosylated hematopoietic stem cells utilized in this figure were transplanted along with a hematopoietic enhancer (NEULASTA®, Amgen, Inc., Thousand Oaks, CA).

### NON-LIMITING ILLUSTRATIVE EMBODIMENTS

Illustrative embodiment 1. A medicament, comprising: a first composition comprising a therapeutically effective amount of at least one ex vivo fucosylated stem cell type; and a second composition comprising a therapeutically effective amount of at least one hematopoietic enhancer, wherein the hematopoietic enhancer is selected from the group consisting of a red cell enhancer, a platelet enhancer, and combinations thereof.

Illustrative embodiment 2. A medicament, comprising: a first composition comprising a therapeutically effective amount of at least one ex vivo fucosylated stem cell type; and a second composition comprising at least one monoclonal antibody selected from the group consisting of Elranatamab, Talquetamab, Epcoritamab, Glofitamab, Ublituximab, Mirvetuximab soravtansine, Nirsevimab, Tremelimumab, Teclistamab, Mosunetuzumab, Relatlimab, Tebentafusp, Tisotumab vedotin, Amivantamab, Loncastuximab tesirine, Margetuximab, Naxitamab, Belantamab mafodotin, Tafasitamab, Sacituzumab govitecan, Isatuximab, [fam]-trastuzumab deruxtecan, Enfortumab vedotin, Polatuzumab vedotin, Moxetumomab pasudotox, Mogamulizumab, Inotuzumab, ozogamicin, Olaratumab, Elotuzumab, Necitumumab, Dinutuximab, Blinatumomab, Ramucirumab, Obinutuzumab, Ado-trastuzumab emtansine, Pertuzumab, Brentuximab vedotin, Ofatumumab, Panitumumab, Bevacizumab, Cetuximab, Panitumumab, Bevacizumab, Cetuximab, Tositumomab, Ibritumomab tiuxetan, Alemtuzumab, Gemtuzumab, Trastuzumab, Rituximab, Edrecolomab, and combinations thereof.

Illustrative embodiment 3. A medicament, comprising: (a) a first composition comprising a therapeutically effective amount of at least one ex vivo fucosylated stem cell type; and at least one (or both) of (a) and (b), wherein (a) is a second composition comprising a therapeutically effective amount of at least one hematopoietic enhancer, wherein the hematopoietic enhancer is selected from the group consisting of a red cell enhancer, a platelet enhancer, and combinations thereof; and (b) is a second composition comprising at least one monoclonal antibody selected from the group consisting of Elranatamab, Talquetamab, Epcoritamab, Glofitamab, Ublituximab, Mirvetuximab soravtansine, Nirsevimab, Tremelimumab, Teclistamab, Mosunetuzumab, Relatlimab, Tebentafusp, Tisotumab vedotin, Amivantamab, Loncastuximab tesirine, Margetuximab, Naxitamab, Belantamab mafodotin, Tafasitamab, Sacituzumab govitecan, Isatuximab, [fam]-trastuzumab deruxtecan, Enfortumab vedotin, Polatuzumab vedotin, Moxetumomab pasudotox, Mogamulizumab, Inotuzumab, ozogamicin, Olaratumab, Daratumumab, Elotuzumab, Necitumumab, Dinutuximab, Blinatumomab, Ramucirumab, Obinutuzumab, Ado-trastuzumab emtansine, Pertuzumab, Brentuximab vedotin, Ofatumumab, Panitumumab, Bevacizumab, Cetuximab, Panitumumab, Bevacizumab, Cetuximab, Tositumomab, Ibritumomab tiuxetan, Alemtuzumab, Gemtuzumab, Trastuzumab, Rituximab, Edrecolomab, and combinations thereof.

Illustrative embodiment 4. The medicament of any of Illustrative embodiments 1-3, wherein the first composition comprises mononuclear cells that comprise at least one ex vivo fucosylated stem cell type and at least one ex vivo fucosylated accessory cell type, wherein the accessory cell type is selected from the group consisting of cytotoxic T-cells, regulatory T-cells, helper T-cells, NK-cells, B-cells, dendritic cells, macrophages, and combinations thereof.

Illustrative embodiment 5. The medicament of any of Illustrative embodiments 1-4, wherein the at least one ex vivo fucosylated stem cell type comprises hematopoietic stem cells.

Illustrative embodiment 6. The medicament of any of Illustrative embodiments 1-5, wherein the stem cells of the at least one ex vivo fucosylated stem cell type are autologous stem cells.

Illustrative embodiment 7. The medicament of any of Illustrative embodiments 1-5, wherein the stem cells of the at least one ex vivo fucosylated stem cell type are allogeneic stem cells.

Illustrative embodiment 7A. The medicament of Illustrative embodiment 7, wherein the allogeneic stem cells are obtained from a source selected from the group consisting of embryonic tissue, fetal tissue, adult tissue, differentiated somatic cells, induced pluripotent stem cells (iPSCs), and combinations thereof.

Illustrative embodiment 7B. The composition of Illustrative embodiment 7A, wherein the embryonic or fetal tissues comprise cord blood.

Illustrative embodiment 8. The medicament of any of Illustrative embodiments 1-7, wherein at least one of: the white cell enhancer is selected from filgrastim, Pegfilgrastim, and combinations thereof; the red cell enhancer is selected from Erythropoietin (EPO), Darbepoietin (dEPO), erythropoiesis-stimulating agents (ESAs), EPO-based constructs (EPO-Fc and methoxy polyethylene glycol-epoetin beta), continuous erythropoietin receptor activator (CERA), peginesatide, EPO-mimetic agents and their constructs, and combinations thereof; and/or the platelet enhancer is selected from thrombopoetin (TPO), romiplostim, eltrombopag, avatrombopag, lusutrombopag, and combinations thereof.

Illustrative embodiment 9. The medicament of any of illustrative embodiments 1-8, wherein the hematopoietic enhancer is not filgrastim.

Illustrative embodiment 10. The medicament of any of Illustrative embodiments 1-8, wherein the second composition comprises romiplostim and filgrastim.

Illustrative embodiment 11. The medicament of any of Illustrative embodiments 1-10, wherein the hematopoietic enhancer is fucosylated ex vivo.

Illustrative embodiment 12. The medicament of Illustrative embodiment 11, wherein the second composition comprises fucosylated filgrastim and fucosylated romiplostim.

Illustrative embodiment 13. The medicament of any of Illustrative embodiments 1-12, wherein the second composition comprises a therapeutically effective amount of at least two hematopoietic enhancers.

Illustrative embodiment 14. The medicament of any of Illustrative embodiments 1-13, wherein the second composition comprises a therapeutically effective amount of at least three hematopoietic enhancers.

Illustrative embodiment 15. The medicament of any of Illustrative embodiments 1-14, wherein the second composition comprises a therapeutically effective amount of at least four hematopoietic enhancers.

Illustrative embodiment 16. The medicament of any of Illustrative embodiments 1-15, wherein the second composition comprises a therapeutically effective amount of at least five hematopoietic enhancers.

Illustrative embodiment 17. The medicament of any of Illustrative embodiments 1-12, wherein the second composition comprises a therapeutically effective amount of at least one red cell enhancer, at least one white cell enhancer, and at least one platelet enhancer.

Illustrative embodiment 18. The medicament of any of Illustrative embodiments 1-17, further comprising a third composition comprising at least one immune cell type selected from the group consisting of stem cells, cytotoxic T-cells, regulatory T-cells, helper T-cells, NK-cells, B-cells, dendritic cells, macrophages, and combinations thereof.

Illustrative embodiment 18A. The medicament of Illustrative embodiment 18, wherein at least one of: the T-cells have been genetically modified to improve their targeting, activation, or production of cytokines; and/or the at least one immune cell type has been ex vivo fucosylated.

Illustrative embodiment 19. A kit, comprising: any portion of the medicament of any of Illustrative embodiments 1-18.

Illustrative embodiment 19A. The kit of Illustrative embodiment 19, wherein each of the first and second compositions is disposed in an IV bag for delivery to the patient.

Illustrative embodiment 19B. The kit of Illustrative embodiment 19A, wherein the first and second compositions are disposed in the same IV bag.

Illustrative embodiment 19C. The kit of Illustrative embodiment 19A, wherein the first and second compositions are disposed in different IV bags.

Illustrative embodiment 19D. The kit of any of Illustrative embodiments 19-19C, wherein the stem cells are present in the IV bag at a concentration of about 10⁷/100 ml.

Illustrative embodiment 19E. The kit of any of Illustrative embodiments 19-19D, further comprising GDP-fucose and an effective amount of a fucosyltransferase (FUT) or an active recombinant fragment of a fucosyltransferase (rFUT).

Illustrative embodiment 19F. The kit of Illustrative embodiment 19E, wherein the FUT is selected from the group consisting of FUT1, FUT2, FUT3, FUT4, FUT5, FUT6, FUT7, FUT8, FUT9, FUT10, FUT11, rFUT1, rFUT2, rFUT3, rFUT4, rFUT5, rFUT6, rFUT7, rFUT8, rFUT9, rFUT10, and rFUT11.

Illustrative embodiment 19G. The kit of Illustrative embodiment 19E or 19F, wherein the rFUT is produced in a mammalian, insect, bacterial, yeast, or fungal expression system and purified prior to contact with the hematopoietic enhancer and GDP-fucose.

Illustrative embodiment 19H. The kit of any of Illustrative embodiments 19E-19G, wherein the rFUT is in a soluble form.

Illustrative embodiment 191. The kit of any of Illustrative embodiments 19E-19H, wherein the rFUT does not contain a membrane binding portion of the native FUT.

Illustrative embodiment 19J. The kit of any of Illustrative embodiments 19-191, further defined as comprising filgrastim in PSB at a concentration in a range of from about 1 to about 5 mg/ml.

Illustrative embodiment 19K. The kit of any of Illustrative embodiments 19-19J, further defined as comprising epoetin alfa (EPOGEN®, Amgen, Inc.) in PSB at a concentration in a range of from about 1,000 to about 2,000 units/ml.

Illustrative embodiment 19L. The kit of any of Illustrative embodiments 19-19K, further defined as comprising Romiplostim at a concentration in a range of from about 5 to about 10 mg/ml.

Illustrative embodiment 20. A method of treating a condition in a patient in need of treatment, the method comprising the step of: administering to the patient the medicament of any of illustrative embodiments 1-18, wherein the first composition is administered simultaneously or wholly or partially sequentially with the second composition.

Illustrative embodiment 21. A method of treating a condition in a patient in need of treatment, the method comprising the steps of: administering, simultaneously or wholly or partially sequentially, to a patient: a first composition comprising a therapeutically effective amount of at least one ex vivo fucosylated stem cell type; and a second composition comprising a therapeutically effective amount of at least one hematopoietic enhancer.

Illustrative embodiment 22. A method of treating a condition in a patient in need of treatment, the method comprising the steps of: administering, simultaneously or wholly or partially sequentially, to a patient: a first composition comprising a therapeutically effective amount of at least one ex vivo fucosylated stem cell type; and a second composition comprising at least one monoclonal antibody selected from the group consisting of Elranatamab, Talquetamab, Epcoritamab, Glofitamab, Ublituximab, Mirvetuximab soravtansine, Nirsevimab, Tremelimumab, Teclistamab, Mosunetuzumab, Relatlimab, Tebentafusp, Tisotumab vedotin, Amivantamab, Loncastuximab tesirine, Margetuximab, Naxitamab, Belantamab mafodotin, Tafasitamab, Sacituzumab govitecan, Isatuximab, [fam]-trastuzumab deruxtecan, Enfortumab vedotin, Polatuzumab vedotin, Moxetumomab pasudotox, Mogamulizumab, Inotuzumab, ozogamicin, Olaratumab, Daratumumab, Elotuzumab, Necitumumab, Dinutuximab, Blinatumomab, Ramucirumab, Obinutuzumab, Ado-trastuzumab emtansine, Pertuzumab, Brentuximab vedotin, Ofatumumab, Panitumumab, Bevacizumab, Cetuximab, Panitumumab, Bevacizumab, Cetuximab, Tositumomab, Ibritumomab tiuxetan, Alemtuzumab, Gemtuzumab, Trastuzumab, Rituximab, Edrecolomab, and combinations thereof.

Illustrative embodiment 23. A method of treating a condition in a patient in need of treatment, the method comprising the steps of: administering, simultaneously or wholly or partially sequentially, to a patient: a first composition comprising a therapeutically effective amount of at least one ex vivo fucosylated stem cell type; and at least one (or both) of (a) and (b), wherein (a) is a second composition comprising a therapeutically effective amount of at least one hematopoietic enhancer, wherein the hematopoietic enhancer is selected from the group consisting of a red cell enhancer, a platelet enhancer, and combinations thereof; and (b) is a second composition comprising at least one monoclonal antibody selected from the group consisting of Elranatamab, Talquetamab, Epcoritamab, Glofitamab, Ublituximab, Mirvetuximab soravtansine, Nirsevimab, Tremelimumab, Teclistamab, Mosunetuzumab, Relatlimab, Tebentafusp, Tisotumab vedotin, Amivantamab, Loncastuximab tesirine, Margetuximab, Naxitamab, Belantamab mafodotin, Tafasitamab, Sacituzumab govitecan, Isatuximab, [fam]-trastuzumab deruxtecan, Enfortumab vedotin, Polatuzumab vedotin, Moxetumomab pasudotox, Mogamulizumab, Inotuzumab, ozogamicin, Olaratumab, Daratumumab, Elotuzumab, Necitumumab, Dinutuximab, Blinatumomab, Ramucirumab, Obinutuzumab, Ado-trastuzumab emtansine, Pertuzumab, Brentuximab vedotin, Ofatumumab, Panitumumab, Bevacizumab, Cetuximab, Panitumumab, Bevacizumab, Cetuximab, Tositumomab, Ibritumomab tiuxetan, Alemtuzumab, Gemtuzumab, Trastuzumab, Edrecolomab, and combinations thereof.

Illustrative embodiment 24. A method, comprising the steps of: (1) identifying a cancer patient in need of a hematopoietic stem cell transplant (HSCT); (2) exposing the cancer patient to an immune ablation conditioning regimen comprising an immunosuppressant; (3) harvesting stem cells from the cancer patient; (4) expanding the stem cells; (5) fucosylating the stem cells by contacting the stem cells with a fucosyltransferase and GDP-fucose to provide a first composition; (6) infusing the first composition comprising fucosylated stem cells into the patient in an amount in a range of from about 10⁵ to about 10⁷ total nucleated cells/kg patient and a minimum of about 3×10⁶ CD4 cells/kg patient; and (7) administering a second composition comprising at least one of a white cell enhancer, a platelet enhancer, and/or a red cell enhancer to the patient simultaneously with the fucosylated stem cells or within 24 hours of administration of the fucosylated stem cells.

Illustrative embodiment 25. A method, comprising the steps of: (1) identifying a cancer patient in need of a hematopoietic stem cell transplant (HSCT); (2) exposing the cancer patient to an immune ablation conditioning regimen comprising an immunosuppressant; (3) harvesting stem cells from the cancer patient; (4) expanding the stem cells; (5) fucosylating the stem cells by contacting the stem cells with a fucosyltransferase and GDP-fucose to provide a first composition; (6) infusing the first composition comprising the fucosylated stem cells into the patient in an amount in a range of from about 10⁵ to about 10⁷ total nucleated cells/kg patient and a minimum of about 3×10⁶ CD4 cells/kg patient; and (7) administering a second composition to the patient simultaneously with the fucosylated stem cells or within 24 hours of administration of the fucosylated stem cells, wherein the second composition comprises a therapeutically effective amount of at least one monoclonal antibody selected from the group consisting of Elranatamab, Talquetamab, Epcoritamab, Glofitamab, Ublituximab, Mirvetuximab soravtansine, Nirsevimab, Tremelimumab, Teclistamab, Mosunetuzumab, Relatlimab, Tebentafusp, Tisotumab vedotin, Amivantamab, Loncastuximab tesirine, Margetuximab, Naxitamab, Belantamab mafodotin, Tafasitamab, Sacituzumab govitecan, Isatuximab, [fam]-trastuzumab deruxtecan, Enfortumab vedotin, Polatuzumab vedotin, Moxetumomab pasudotox, Mogamulizumab, Inotuzumab, ozogamicin, Olaratumab, Daratumumab, Elotuzumab, Necitumumab, Dinutuximab, Blinatumomab, Ramucirumab, Obinutuzumab, Ado-trastuzumab emtansine, Pertuzumab, Brentuximab vedotin, Ofatumumab, Panitumumab, Bevacizumab, Cetuximab, Panitumumab, Bevacizumab, Cetuximab, Tositumomab, Ibritumomab tiuxetan, Alemtuzumab, Gemtuzumab, Trastuzumab, Rituximab, Edrecolomab, and combinations thereof.

Illustrative embodiment 25A. The method of Illustrative embodiment 24 or 25, wherein the immunosuppressant of step (2) comprises tacrolimus.

Illustrative embodiment 26. The method of any of Illustrative embodiments 21-25, wherein the first composition comprises mononuclear cells that comprise at least one ex vivo fucosylated stem cell type and at least one ex vivo fucosylated accessory cell type, wherein the accessory cell type is selected from the group consisting of cytotoxic T-cells, regulatory T-cells, helper T-cells, NK-cells, B-cells, dendritic cells, macrophages, and combinations thereof.

Illustrative embodiment 27. The method of any of Illustrative embodiments 21-26, wherein the at least one ex vivo fucosylated stem cell type comprises hematopoietic stem cells.

Illustrative embodiment 28. The method of any of Illustrative embodiments 21-27, wherein the stem cells of the at least one ex vivo fucosylated stem cell type are autologous stem cells.

Illustrative embodiment 29. The method of any of Illustrative embodiments 21-27, wherein the stem cells of the at least one ex vivo fucosylated stem cell type are allogeneic stem cells.

Illustrative embodiment 29A. The method of Illustrative embodiment 29, wherein the allogeneic stem cells are obtained from a source selected from the group consisting of embryonic tissue, fetal tissue, adult tissue, differentiated somatic cells, induced pluripotent stem cells (iPSCs), and combinations thereof.

Illustrative embodiment 29B. The method of Illustrative embodiment 29A, wherein the embryonic or fetal tissues comprise cord blood.

Illustrative embodiment 30. The method of any of Illustrative embodiments 21-29B, wherein the hematopoietic enhancer is selected from the group consisting of a red cell enhancer, a white cell enhancer, a platelet enhancer, and combinations thereof.

Illustrative embodiment 31. The method of any of Illustrative embodiments 21-30, wherein at least one of: the white cell enhancer is selected from filgrastim, Pegfilgrastim, and combinations thereof; the red cell enhancer is selected from Erythropoietin (EPO), Darbepoietin (dEPO), erythropoiesis-stimulating agents (ESAs), EPO-based constructs (EPO-Fc and methoxy polyethylene glycol-epoetin beta), continuous erythropoietin receptor activator (CERA), peginesatide, EPO-mimetic agents and their constructs, and combinations thereof; and/or the platelet enhancer is selected from thrombopoetin (TPO), romiplostim, eltrombopag, avatrombopag, lusutrombopag, and combinations thereof.

Illustrative embodiment 32. The method of any of illustrative embodiments 21-31, wherein the hematopoietic enhancer is not filgrastim.

Illustrative embodiment 33. The method of any of Illustrative embodiments 21-31, wherein the second composition comprises romiplostim and filgrastim.

Illustrative embodiment 34. The method of any of Illustrative embodiments 21-33, wherein the hematopoietic enhancer is ex vivo fucosylated.

Illustrative embodiment 35. The method of Illustrative embodiment 34, wherein the second composition comprises fucosylated filgrastim and fucosylated romiplostim.

Illustrative embodiment 36. The method of any of Illustrative embodiments 21-35, wherein the second composition comprises a therapeutically effective amount of at least two hematopoietic enhancers.

Illustrative embodiment 37. The method of any of Illustrative embodiments 21-36, wherein the second composition comprises a therapeutically effective amount of at least three hematopoietic enhancers.

Illustrative embodiment 38. The method of any of Illustrative embodiments 21-37, wherein the second composition comprises a therapeutically effective amount of at least four hematopoietic enhancers.

Illustrative embodiment 39. The method of any of Illustrative embodiments 21-38, wherein the second composition comprises a therapeutically effective amount of at least five hematopoietic enhancers.

Illustrative embodiment 40. The method of any of Illustrative embodiments 21-39, wherein the second composition comprises a therapeutically effective amount of at least one red cell enhancer, at least one white cell enhancer, and at least one platelet enhancer.

Illustrative embodiment 41. The method of any of Illustrative embodiments 21-40, further comprising the step of administering a third composition to the patient, wherein the third composition is administered simultaneously or wholly or partially sequentially with the first and/or second compositions, and wherein the third composition comprises at least one immune cell type selected from the group consisting of stem cells, cytotoxic T-cells, regulatory T-cells, helper T-cells, NK-cells, B-cells, dendritic cells, macrophages, and combinations thereof.

Illustrative embodiment 41A. The method of Illustrative embodiment 41, wherein at least one of: the T-cells have been genetically modified to improve their targeting, activation, or production of cytokines; and/or the at least one immune cell type has been ex vivo fucosylated.

Illustrative embodiment 42. The method of any of Illustrative embodiments 21-41, wherein the first composition is administered contemporaneously with the at least one second composition.

Illustrative embodiment 43. The method of any of Illustrative embodiments 21-41, wherein the at least one second composition is administered within about 24 hours after administration of the first composition.

Illustrative embodiment 44. The method of any of Illustrative embodiments 21-43, wherein the step of administering the second composition is repeated one or more times.

Illustrative embodiment 45. The method of any of Illustrative embodiments 21-44, further defined as a method of reducing at least one adverse event during HSCT therapy, wherein the adverse event is selected from the group consisting of an infection, Graft versus Host Disease (GvHD), internal bleeding, and graft rejection.

Illustrative embodiment 46. The method of any of Illustrative embodiments 21-45, wherein the first composition is infused to the patient at a dose of from about 10⁵ to about 10⁷ stem cells/kg.

Illustrative embodiment 46A. The method of Illustrative embodiment 46, wherein the first composition is infused to the patient at a dose of about 10⁶ stem cells/kg.

Illustrative embodiment 46B. The method of any of Illustrative embodiments 21-46A, wherein the first composition is infused to the patient in a liquid (1 L) comprising about 0.5% to about 5% fucose.

Illustrative embodiment 46C. The method of any of Illustrative embodiments 21-46B, wherein filgrastim is infused to the patient at a dose in a range of from about 5 to about 10 μg/kg/day, and wherein romiplostim is infused to the patient at a dose in a range of from about 0.5 to about 1.5 μg/kg/day.

Illustrative embodiment 46D. The method of any of Illustrative embodiments 21-46C, wherein the second composition is administered to the patient on a daily basis for a period in a range of from about 1 day to about 21 days.

Illustrative embodiment 47. The method of any of Illustrative embodiments 21-46D, wherein the patient is a cancer patient, and wherein the method is further defined as a method of treating cancer.

Illustrative embodiment 47A. The method of Illustrative embodiment 47, wherein the cancer is selected from the group consisting of prostate cancer, skin cancer, ovarian cancer, a cancer of a non-lymphoid parenchymal organ, breast cancer, a cancer of the head and neck, mantle cell lymphoma, Non-Hodgkin B cell lymphoma, PTCL, adenoma, squamous cell carcinoma, laryngeal carcinoma, salivary carcinoma, thymoma, thymic carcinoma, leukemia, retinal cancer, esophageal cancer, multiple myeloma, melanoma, colorectal cancer, lung cancer, cervical cancer, endometrium carcinoma, gallbladder cancer, liver cancer, thyroid follicular cancer, gastric cancer, non-small cell lung carcinoma, glioma, urothelial cancer, bladder cancer, prostate cancer, renal cell cancer, infiltrating ductal carcinoma, glioblastoma multiform, and combinations thereof.

Illustrative embodiment 48. The method of Illustrative embodiment 47 or 47A, wherein the cancer is a hematopoietic cancer.

Illustrative embodiment 48A. The method of Illustrative embodiment 48, wherein the hematopoietic cancer is selected from the group consisting of leukemia, lymphoma, multiple myeloma, Acute myeloid (or myelogenous) leukemia (AML), Chronic myeloid (or myelogenous) leukemia (CML), Acute lymphocytic (or lymphoblastic) leukemia (ALL), Chronic lymphocytic leukemia (CLL), Hodgkin lymphoma, non-Hodgkin lymphoma (NHL), Light Chain Myeloma, Non-secretory Myeloma, Solitary Plasmacytoma, Extramedullary Plasmacytoma, Monoclonal Gammopathy of Undetermined Significance (MGUS), Smoldering Multiple Myeloma (SMM), Immunoglobulin D (IgD) Myeloma, Immunoglobulin E (IgE) Myeloma, and combinations thereof.

Illustrative embodiment 49. The method of any of Illustrative embodiments 21-48A, wherein the method is further defined as a method of treating an autoimmune disease.

Illustrative embodiment 49A. The method of Illustrative embodiment 49, wherein the autoimmune disease is selected from the group consisting of Acfatigueromegaly, Acquired aplastic anemia, Acquired hemophilia, Agammaglobulinemia, primary, Alopecia areata, Ankylosing spondylitis (AS), Anti-NMDA receptor encephalitis, Antiphospholipid syndrome (APS) (catastrophic antiphospholipid syndrome (CAPS), Asherson's syndrome), Arteriosclerosis, Autoimmune Addison's disease (AAD), Autoimmune autonomic ganglionopathy (AAG) (autoimmune dysautonomia, autoimmune gastrointestinal dysmotility (AGID)), Autoimmune encephalitis (acute disseminated encephalomyelitis (ADEM)), Autoimmune gastritis, Autoimmune hemolytic anemia (AIHA), Autoimmune hepatitis (AIH), Autoimmune hyperlipidemia, Autoimmune hypophysitis, Autoimmune inner ear disease (AIED), Autoimmune lymphoproliferative syndrome (ALPS). Autoimmune myelofibrosis, Autoimmune myocarditis, Autoimmune oophoritis, Autoimmune pancreatitis (AIP), Autoimmune polyglandular syndromes, types I, II, & III (APS type 1, APS type 2, APS type 3, APECED), Autoimmune progesterone dermatitis, *Autoimmune retinopathy (AIR), *Autoimmune sudden sensorineural hearing loss (SNHL), Balo disease, Behçet's disease, Birdshot chorioretinopathy/birdshot uveitis, Bullous pemphigoid, Castleman disease, Celiac disease, Chagas disease, Chronic inflammatory demyelinating polyneuropathy (CIDP), Chronic urticaria (CU), Churg-Strauss syndrome/eosinophilic granulomatosis with polyangiitis (EGPA), Cogan's syndrome, Cold agglutinin disease, CREST syndrome|limited cutaneous systemic sclerosis, Crohn's disease (CD), Cronkhite-Canada syndrome (CSS), Cryptogenic organizing pneumonia (COP), Dermatitis herpetiformis, Dermatomyositis, Devic's disease/neuromyelitis optica (NMO), Diabetes, type 1, Discoid lupus, Dressler's syndrome/postmyocardial infarction/postpericardiotomy syndrome, Eczema/Atopic Dermatitis, Endometriosis, Eosinophilic esophagitis, Eosinophilic fasciitis, Erythema nodosum, Essential mixed cryoglobulinemia, Evans syndrome, Fibrosing alveolitis/Idiopathic pulmonary fibrosis (IPF), Giant cell arteritis/temporal arteritis/Horton's disease, Glomerulonephritis, Goodpasture's syndrome/anti-GBM/anti-TBM disease, Granulomatosis with polyangiitis (GPA)/Wegener's granulomatosis, Graves' disease/thyroid eye disease, Guillain-Barré syndrome (GBS), Hashimoto's thyroiditis/chronic lymphocytic thyroiditis/autoimmune thyroiditis, Henoch-Schönlein purpura/IgA vasculitis, Hidradenitis suppurativa, Hurst's disease/acute hemorrhagic leukoencephalitis (AHLE), Hypogammaglobulinemia, IgA nephropathy/Berger's disease, Immune-mediated necrotizing myopathy (IMNM), Immune thrombocytopenia (ITP)/autoimmune thrombocytopeniarpura/autoimmune thrombocytopenia, Inclusion body myositis, IgG4-related sclerosing disease (ISD), Interstitial cystitis, Juvenile idiopathic arthritis/Adult-onset Still's disease, Juvenile polymyositis/Juvenile dermatomyositis/juvenile myositis, Kawasaki disease, Lambert-Eaton myasthenic syndrome (LEMS), Leukocytoclastic vasculitis, Lichen planus, Lichen sclerosus, Ligneous conjunctivitis, Linear IgA disease (LAD)/linear IgA bullous dermatosis (LABD), Lupus nephritis, Lyme disease/chronic Lyme disease/post-treatment Lyme disease syndrome (PTLDS), Lymphocytic colitis/microscopic colitis, Ménière's disease, Microscopic polyangiitis (MPA)/ANCA-associated vasculitis, Mixed connective tissue disease (MCTD), Mooren's ulcer, Mucha-Habermann disease, Multifocal motor neuropathy, Multiple sclerosis (MS), Myalgic encephalomyelitis (ME)/Chronic fatigue syndrome (CFS), Myasthenia gravis (MG), Narcolepsy, Ocular cicatricial pemphigoid, Opsoclonus-myoclonus syndrome (OMS), Palindromic rheumatism, Paraneoplastic cerebellar degeneration, Paraneoplastic pemphigus, Parry-Romberg syndrome (PRS)/Hemifacial atrophy (HFA)/Progressive facial hemiatrophy, Paroxysmal nocturnal hemoglobinuria (PNH), Peripheral uveitis/pars planitis, PANS/PANDAS, Parsonage-Turner syndrome, Pemphigus gestationis/herpes gestationis, Pemphigus foliaceus, Pemphigus vulgaris, Pernicious anemia, POEMS syndrome, Polyarteritis nodosa, Polymyalgia rheumatica, Polymyositis, Postural orthostatic tachycardia syndrome (POTS), Primary biliary cirrhosis (PBC)/primary biliary cholangitis, Primary sclerosing cholangitis (PSC), Psoriasis, Palmoplantar Pustulosis, Psoriatic arthritis, Pulmonary fibrosis, idiopathic (IPF), Pure red cell aplasia (PRCA), Pyoderma gangrenosum, Raynaud's syndrome/phenomenon, Reactive arthritis/Reiter's syndrome, Reflex sympathetic dystrophy syndrome (RSD)/Complex regional pain syndrome (CRPS), Relapsing polychondritis, Restless leg syndrome (RLS)/Willis-Ekbom disease; Rheumatic fever, Rheumatoid arthritis, Sarcoidosis, Schmidt syndrome/autoimmune polyendocrine syndrome type II, Scleritis, Scleroderma, Serpiginous choroidopathy, Sjögren's syndrome, Stiff person syndrome (SPS), Small fiber sensory neuropathy, Systemic lupus erythematosus (SLE), Subacute bacterial endocarditis (SBE), Susac syndrome, Sydenham's chorea, Sympathetic ophthalmia, Takayasu's arteritis (vasculitis), Testicular autoimmunity (vasculitis, orchitis), Tolosa-Hunt syndrome, Transverse myelitis (TM); Tubulointerstitial nephritis uveitis syndrome (TINU), Ulcerative colitis (UC); Undifferentiated connective tissue disease (UCTD), Uveitis/anterior/intermediate/posterior, Vasculitis, VEXAS Syndrome, Vitiligo, Vogt-Koyanagi-Harada syndrome (VKH), and combinations thereof.

Illustrative embodiment 50. The method of any of Illustrative embodiments 21-49A, wherein the method is further defined as a method of treating a non-cancerous blood disorder.

Illustrative embodiment 50A. The method of Illustrative embodiment 50, wherein the non-cancerous blood disorder is sickle cell disease or a thalassemia.

Illustrative embodiment 51. The method of any of Illustrative embodiments 21-50A, wherein the white cell enhancer is administered to the patient at a dosage in a range of from about 5 μg/kg/day to about 10 μg/kg/day until the patient's neutrophil count achieves 0.5×10⁹/L.

Illustrative embodiment 52. The method of any of Illustrative embodiments 21-51, wherein the platelet enhancer is administered to the patient at a dosage of about 10 μg/kg/day until the patient's platelet count achieves 20×10⁹/L.

Illustrative embodiment 53. The method of any of Illustrative embodiments 21-52, wherein the red cell enhancer is administered to the patient at a dosage in a range of from about 20 units/kg/day to about 50 units/kg/day until the patient's red Hbg level achieves 10 g/dL.

Illustrative embodiment 54. A method of increasing engraftment of hematopoietic stem cells and myeloid lineage cells upon hematopoietic stem cell therapy with mononuclear cells, the method comprising the steps of: (1) identifying a cancer patient in need of a hematopoietic stem cell transplant (HSCT); (2) exposing the cancer patient to an immune ablation conditioning regimen comprising an immunosuppressant; (3) harvesting mononuclear cells from the cancer patient; (4) expanding the mononuclear cells; (5) fucosylating at least a portion of the mononuclear cells by contacting the mononuclear cells with a fucosyltransferase and GDP-fucose; and (6) infusing the fucosylated mononuclear cells into the patient in an amount in a range of from about 10⁵ to about 10⁷ total nucleated cells/kg patient and a minimum of about 3×10⁶ CD4 cells/kg patient; and wherein hematopoietic stem cells and myeloid lineage cells exhibit increased engraftment when compared to engraftment of hematopoietic stem cells and myeloid lineage cells following infusion of non-fucosylated mononuclear cells.

Illustrative embodiment 55. The method of Illustrative embodiment 54, wherein the first composition comprises mononuclear cells that comprise at least one ex vivo fucosylated stem cell type and at least one ex vivo fucosylated accessory cell type, wherein the accessory cell type is selected from the group consisting of cytotoxic T-cells, regulatory T-cells, helper T-cells, NK-cells, B-cells, dendritic cells, macrophages, and combinations thereof.

Illustrative embodiment 56. The method of Illustrative embodiment 54 or 55, wherein the at least one ex vivo fucosylated stem cell type comprises hematopoietic stem cells.

Illustrative embodiment 57. The method of any of Illustrative embodiments 54-56, wherein the stem cells of the at least one ex vivo fucosylated stem cell type are autologous stem cells.

Illustrative embodiment 58. The method of any of Illustrative embodiments 54-57, wherein the stem cells of the at least one ex vivo fucosylated stem cell type are allogeneic stem cells.

Illustrative embodiment 59. The method of any of Illustrative embodiments 54-58, further comprising the step of administering at least one additional composition simultaneously or wholly or partially sequentially with step (6), and wherein the at least one additional composition comprises at least one of: a white cell enhancer; a platelet enhancer; a red cell enhancer; and/or at least one monoclonal antibody selected from the group consisting of Elranatamab, Talquetamab, Epcoritamab, Glofitamab, Ublituximab, Mirvetuximab soravtansine, Nirsevimab, Tremelimumab, Teclistamab, Mosunetuzumab, Relatlimab, Tebentafusp, Tisotumab vedotin, Amivantamab, Loncastuximab tesirine, Margetuximab, Naxitamab, Belantamab mafodotin, Tafasitamab, Sacituzumab govitecan, Isatuximab, [fam]-trastuzumab deruxtecan, Enfortumab vedotin, Polatuzumab vedotin, Moxetumomab pasudotox, Mogamulizumab, Inotuzumab, ozogamicin, Olaratumab, Daratumumab, Elotuzumab, Necitumumab, Dinutuximab, Blinatumomab, Ramucirumab, Obinutuzumab, Ado-trastuzumab emtansine, Pertuzumab, Brentuximab vedotin, Ofatumumab, Panitumumab, Bevacizumab, Cetuximab, Panitumumab, Bevacizumab, Cetuximab, Tositumomab, Ibritumomab tiuxetan, Alemtuzumab, Gemtuzumab, Trastuzumab, Rituximab, Edrecolomab, and combinations thereof.

Illustrative embodiment 60. The method of Illustrative embodiment 59, wherein the at least one additional composition is administered simultaneously with step (6).

Illustrative embodiment 61. The method of Illustrative embodiment 59, wherein the at least one additional composition is administered within about 24 hours after administration of the first composition.

Illustrative embodiment 62. The method of any of Illustrative embodiments 59-61, wherein the step of administering the at least one composition is repeated one or more times.

Illustrative embodiment 63. The method of any of Illustrative embodiments 59-62, wherein at least one of: the white cell enhancer is selected from filgrastim, Pegfilgrastim, and combinations thereof; the red cell enhancer is selected from Erythropoietin (EPO), Darbepoietin (dEPO), erythropoiesis-stimulating agents (ESAs), EPO-based constructs (EPO-Fc and methoxy polyethylene glycol-epoetin beta), continuous erythropoietin receptor activator (CERA), peginesatide, EPO-mimetic agents and their constructs, and combinations thereof; and/or the platelet enhancer is selected from thrombopoetin (TPO), romiplostim, eltrombopag, avatrombopag, lusutrombopag, and combinations thereof.

Illustrative embodiment 64. The method of any of illustrative embodiments 59-63, wherein the hematopoietic enhancer is not filgrastim.

Illustrative embodiment 65. The method of any of Illustrative embodiments 59-63, wherein the at least one additional composition comprises romiplostim and filgrastim.

Illustrative embodiment 66. The method of any of Illustrative embodiments 59-65, wherein the hematopoietic enhancer is ex vivo fucosylated.

Illustrative embodiment 67. The method of Illustrative embodiment 66, wherein the at least one additional composition comprises fucosylated filgrastim and fucosylated romiplostim.

Illustrative embodiment 68. The method of any of Illustrative embodiments 59-67, wherein the at least one additional composition comprises a therapeutically effective amount of at least two hematopoietic enhancers.

Illustrative embodiment 69. The method of any of Illustrative embodiments 59-68, wherein the at least one additional composition comprises a therapeutically effective amount of at least three hematopoietic enhancers.

Illustrative embodiment 70. The method of any of Illustrative embodiments 59-69, wherein the at least one additional composition comprises a therapeutically effective amount of at least four hematopoietic enhancers.

Illustrative embodiment 71. The method of any of Illustrative embodiments 59-70, wherein the at least one additional composition comprises a therapeutically effective amount of at least five hematopoietic enhancers.

Illustrative embodiment 72. The method of any of Illustrative embodiments 59-71, wherein the at least one additional composition comprises a therapeutically effective amount of at least one red cell enhancer, at least one white cell enhancer, and at least one platelet enhancer.

Illustrative embodiment 73. The method of any of Illustrative embodiments 59-72, further comprising the step of administering a third composition to the patient, wherein the third composition is administered simultaneously or wholly or partially sequentially with the first and/or second compositions, and wherein the third composition comprises at least one immune cell type selected from the group consisting of stem cells, cytotoxic T-cells, regulatory T-cells, helper T-cells, NK-cells, B-cells, dendritic cells, macrophages, and combinations thereof.

Illustrative embodiment 73A. The method of Illustrative embodiment 73, wherein at least one of: the T-cells have been genetically modified to improve their targeting, activation, or production of cytokines; and/or the at least one immune cell type has been ex vivo fucosylated.

Illustrative embodiment 74. The medicament, kit, or method of any of Illustrative embodiments 1-73, wherein any ex vivo fucosylated cells or hematopoietic enhancers is fucosylated ex vivo by contact with GDP-fucose and an effective amount of a fucosyltransferase (FUT) or an active recombinant fragment of a fucosyltransferase (rFUT).

Illustrative embodiment 74A. The medicament, kit, or method of Illustrative embodiment 74, wherein the FUT is selected from the group consisting of FUT1, FUT2, FUT3, FUT4, FUT5, FUT6, FUT7, FUT8, FUT9, FUT10, FUT11, rFUT1, rFUT2, rFUT3, rFUT4, rFUT5, rFUT6, rFUT7, rFUT8, rFUT9, rFUT10, and rFUT11.

Illustrative embodiment 74B. The medicament, kit, or method of Illustrative embodiment 74 or 74A, wherein the rFUT is produced in a mammalian, insect, bacterial, yeast, or fungal expression system and purified prior to contact with the hematopoietic enhancer and GDP-fucose.

Illustrative embodiment 74C. The medicament, kit, or method of any of Illustrative embodiments 74-74B, wherein the rFUT is in a soluble form.

Illustrative embodiment 74D. The medicament, kit, or method of any of Illustrative embodiments 74-74C, wherein the rFUT does not contain a membrane binding portion of the native FUT.

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

1-61. (canceled)

62. A medicament, comprising:
a first composition comprising a therapeutically effective amount of at least one ex vivo fucosylated stem cell type; and
a second composition comprising a therapeutically effective amount of at least one agent selected from the group consisting of a hematopoietic enhancer, a growth factor, a monoclonal antibody, and combinations thereof, and wherein:
(a) the hematopoietic enhancer is selected from the group consisting of a red cell enhancer, a platelet enhancer, a white cell enhancer, and combinations thereof; and
(b) the monoclonal antibody is selected from the group consisting of Elranatamab, Talquetamab, Epcoritamab, Glofitamab, Ublituximab, Mirvetuximab soravtansine, Nirsevimab, Tremelimumab, Teclistamab, Mosunetuzumab, Relatlimab, Tebentafusp, Tisotumab vedotin, Amivantamab, Loncastuximab tesirine, Margetuximab, Naxitamab, Belantamab mafodotin, Tafasitamab, Sacituzumab govitecan, Isatuximab, [fam]-trastuzumab deruxtecan, Enfortumab vedotin, Polatuzumab vedotin, Moxetumomab pasudotox, Mogamulizumab, Inotuzumab ozogamicin, Olaratumab, Daratumumab, Elotuzumab, Necitumumab, Dinutuximab, Blinatumomab, Ramucirumab, Obinutuzumab, Ado-trastuzumab emtansine, Pertuzumab, Brentuximab vedotin, Ofatumumab, Panitumumab, Bevacizumab, Cetuximab, Tositumomab, Ibritumomab tiuxetan, Alemtuzumab, Gemtuzumab, Trastuzumab, Rituximab, Edrecolomab, and combinations thereof.

63. The medicament of claim 62, wherein the first composition comprises mononuclear cells that comprise at least one ex vivo fucosylated stem cell type and at least one ex vivo fucosylated accessory cell type, wherein the accessory cell type is selected from the group consisting of cytotoxic T-cells, regulatory T-cells, helper T-cells, NK-cells, B-cells, dendritic cells, macrophages, and combinations thereof.

64. The medicament of claim 62, wherein the at least one fucosylated stem cell type comprises hematopoietic stem cells or mesenchymal stem cells.

65. The medicament of claim 62, wherein the stem cells of the at least one fucosylated stem cell type are allogeneic or autologous stem cells.

66. The medicament of claim 62, wherein at least one of:
the red cell enhancer is selected from Erythropoietin (EPO), Darbepoietin (dEPO), erythropoiesis-stimulating agents (ESAs), EPO-based constructs (EPO-Fc and methoxy polyethylene glycol-epoetin beta), continuous erythropoietin receptor activator (CERA), peginesatide, EPO-mimetic agents and their constructs, and combinations thereof;
the platelet enhancer is selected from thrombopoetin (TPO), romiplostim, eltrombopag, avatrombopag, lusutrombopag, and combinations thereof;
the white cell enhancer is Pegfilgrastim; and/or
the growth factor is selected from the group consisting of IL2, IL7, IL8, IL10, IL12, IL15, IL23, and combinations thereof.

67. A kit, comprising the medicament of claim 62.

68. A method of treating a condition in a patient in need of treatment, the method comprising the steps of:
administering to the patient a first composition comprising a therapeutically effective amount of at least one fucosylated stem cell type; and
administering to the patient a second composition comprising a therapeutically effective amount of at least one agent selected from the group consisting of a hematopoietic enhancer, a growth factor, a monoclonal antibody, and combinations thereof, and wherein:
(a) the hematopoietic enhancer is selected from the group consisting of a red cell enhancer, a platelet enhancer, a white cell enhancer, and combinations thereof; and
(b) the monoclonal antibody is selected from the group consisting of Elranatamab, Talquetamab, Epcoritamab, Glofitamab, Ublituximab, Mirvetuximab soravtansine, Nirsevimab, Tremelimumab, Teclistamab, Tebentafusp, Mosunetuzumab, Relatlimab, Tisotumab vedotin, Amivantamab, Loncastuximab tesirine, Margetuximab, Naxitamab, Belantamab mafodotin, Tafasitamab, Sacituzumab govitecan, Isatuximab, [fam]-trastuzumab deruxtecan, Enfortumab vedotin, Polatuzumab vedotin, Moxetumomab pasudotox, Mogamulizumab, Inotuzumab ozogamicin, Olaratumab, Daratumumab, Elotuzumab, Necitumumab, Dinutuximab, Blinatumomab, Ramucirumab, Obinutuzumab, Ado-trastuzumab emtansine, Pertuzumab, Brentuximab vedotin, Ofatumumab, Panitumumab, Bevacizumab, Cetuximab, Tositumomab, Ibritumomab tiuxetan, Alemtuzumab, Gemtuzumab, Trastuzumab, Rituximab, Edrecolomab, and combinations thereof; and
wherein the first and second compositions are administered simultaneously or wholly or partially sequentially.

69. The method of claim 68, wherein the first composition comprises mononuclear cells that comprise at least one ex vivo fucosylated stem cell type and at least one fucosylated accessory cell type, wherein the accessory cell type is selected from the group consisting of cytotoxic T-cells, regulatory T-cells, helper T-cells, NK-cells, B-cells, dendritic cells, macrophages, and combinations thereof.

70. The method of claim 68, wherein the stem cells of the at least one fucosylated stem cell type are allogeneic or autologous stem cells.

71. The method of claim 68, wherein the first composition is infused to the patient at a dose of from about 10⁵ to about 10⁷ stem cells/kg.

72. The method of claim 68, wherein the patient is a cancer patient having a hematopoietic cancer, and wherein the method is further defined as a method of treating cancer.

73. The method of claim 68, further defined as a method of reducing at least one adverse event during hematopoietic stem cell transplantation (HSCT) therapy, wherein the adverse event is selected from the group consisting of an infection, Graft versus Host Disease (GvHD), internal bleeding, graft rejection, and combinations thereof.

74. The method of claim 68, wherein the second composition is administered to the patient contemporaneously with, or within about 24 hours after, administration of the first composition.

75. The method of claim 68, wherein the step of administering the second composition is repeated one or more times.

76. The method of claim 68, wherein the method is further defined as a method of treating an autoimmune disease or a non-cancerous blood disorder.

77. A method of treating a disease in the patient, the method comprising the steps of:
(1) identifying a patient in need of a stem cell transplant;
(2) exposing the patient to an immune ablation conditioning regimen comprising an immunosuppressant;
(3) harvesting mononuclear cells from the patient;
(4) fucosylating at least a portion of the mononuclear cells by contacting the mononuclear cells with a fucosyltransferase and GDP-fucose in the absence of expansion of the mononuclear cells;
(5) infusing the fucosylated mononuclear cells into the patient in an amount in a range of from about 10⁵ to about 10⁷ total nucleated cells/kg patient and a minimum of about 3×10⁶ CD4 cells/kg patient; and
(6) administering a therapeutically effective amount of at least one agent selected from the group consisting of a hematopoietic enhancer, a growth factor, a monoclonal antibody, and combinations thereof, and wherein:
(a) the hematopoietic enhancer is selected from the group consisting of a red cell enhancer, a platelet enhancer, a white cell enhancer, and combinations thereof; and
(b) the monoclonal antibody is selected from the group consisting of Elranatamab, Talquetamab, Epcoritamab, Glofitamab, Ublituximab, Mirvetuximab soravtansine, Nirsevimab, Tremelimumab, Teclistamab, Mosunetuzumab, Relatlimab, Tebentafusp, Tisotumab vedotin, Amivantamab, Loncastuximab tesirine, Margetuximab, Naxitamab, Belantamab mafodotin, Tafasitamab, Sacituzumab govitecan, Isatuximab, [fam]-trastuzumab deruxtecan, Enfortumab vedotin, Polatuzumab vedotin, Moxetumomab pasudotox, Mogamulizumab, Inotuzumab ozogamicin, Olaratumab, Daratumumab, Elotuzumab, Necitumumab, Dinutuximab, Ramucirumab, Obinutuzumab, Ado-trastuzumab emtansine, Pertuzumab, Brentuximab vedotin, Ofatumumab, Panitumumab, Bevacizumab, Cetuximab, Tositumomab, Ibritumomab tiuxetan, Alemtuzumab, Gemtuzumab, Trastuzumab, Rituximab, Edrecolomab, and combinations thereof;
wherein stem cells and myeloid lineage cells in the infusion of fucosylated mononuclear cells exhibit increased engraftment when compared to engraftment of stem cells and myeloid lineage cells following infusion of non-fucosylated mononuclear cells.

78. The method of claim 77, wherein the mononuclear cells harvested in step (3) comprise hematopoietic stem cells and at least one accessory cell type selected from the group consisting of cytotoxic T-cells, regulatory T-cells, helper T-cells, NK-cells, B-cells, dendritic cells, macrophages, and combinations thereof.

79. The method of claim 77, further comprising the step of repeating administration of the at least one agent of step (6) to the patient more than 24 hours after infusion of the fucosylated mononuclear cells in step (5).

80. The method of claim 77, wherein step (6) is further defined as administering at least one white cell enhancer, at least one platelet enhancer, or at least one red cell enhancer to the patient.


SEQUENCE LISTING (1 sequence)

SEQ ID NO: 1 - protein, 359 aa, Homo sapiens [sequence omitted]